(19) United States
(12) Patent Application Publication
(10) Pub. No.: US 2018/0155735 A1
(43) Pub. Date: Jun. 7, 2018

(54) IDENTIFICATION OF TRANSCRIPTION FACTORS THAT IMPROVE NITROGEN AND SULPHUR USE EFFICIENCY IN PLANTS

(71) Applicants: BIOGEMMA, Paris (FR); UNIVERSIDAD POLITECNICA DE MADRID, Madrid (ES); INSTITUTO NACIONAL DE INVESTIGACION Y TECNOLOGIA AGRARIA Y ALIMENTARIA, Madrid (ES)

(72) Inventors: Marcela GOMEZ-PAEZ, Madrid (ES); Jesus VICENTE-CARBAJOSA, Madrid (ES); Joaquin MEDINA, Tres Cantos (ES); Stephane LAFARGE, Chappes (US)

(21) Appl. No.: 15/737,150

(22) PCT Filed: Jun. 15, 2016
(86) PCT No.: PCT/EP2016/063735

§ 371 Date: Dec. 15, 2017

(30) Foreign Application Priority Data
Jun. 15, 2015 (EP) 15305927.4

(51) Int. Cl. C12N 15/82; A01H 5/00; A01H 1/02; C07H 21/04

(52) U.S. Cl. CPC C12N 15/8243; C07H 21/04; A01H 1/02; A01H 5/00

(57) ABSTRACT

The present invention provides an expression cassette comprising at least one polynucleotide comprising a nucleotide sequence selected from the group consisting of: a) a nucleotide sequence that encodes a polypeptide having the amino acid sequence set forth in SEQ ID NOs. 13-24; and b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24. This expression cassette can be used in vivo to increase in plants the tolerance to abiotic stress, such as e.g., sulphur deficiency, nitrogen deficiency, or carbon/nitrogen imbalance. In another aspect, the invention thus relates to a transgenic plant cell comprising the expression cassette described above.

### INTRODUCTION

Plant growth and development are regulated by specific metabolic and signalling pathways, which are precisely elicited by environmental conditions and developmental cues. Nutrient availability and in particular that of carbon (C), nitrogen (N) and sulphur (S) is one of the most important factors for the regulation of plant metabolism. In addition to their independent utilization, the ratio of C to N metabolites in the cell is also important for the regulation of plant growth, and is referred to as the “C/N balance” (Coruzzi and Zhou 2001, Martin et al. 2002). In natural conditions the availability of C, N and S changes in relation to environmental factors like drought, rainfall, light availability, atmospheric CO₂, diurnal cycles, etc. (Gibon et al. 2004, Miller et al. 2007, Smith and Stitt 2007, Kiba et al. 2011). Moreover, extreme temperatures and pathogens might also promote major changes in carbohydrate partitioning and metabolism (Klotke et al. 2004, Roitsch and Gonzalez 2004). In response to those environmental challenges, plants have developed specialized molecular mechanisms to perceive and respond to unbalance nutrient conditions by promoting accurate partitioning of C, N and S sources and setting a complex metabolic rearrangement (Sato et al. 2011b, Sulpice et al. 2013).

In the last years major efforts have been made to develop new crop varieties with improved nutrient assimilation efficiencies. However, limited success has been achieved since breeding programmes had to deal with complex traits with polygenic inheritance (Fernandez-Muñoz 2005, Paterson et al, 1988, Ribaut et al, 2010, Wang et al, 2012, Foolad et al, 2007). Initial attempts to develop plants of agronomic value with improved nutrient assimilation efficiencies were based on genetic transformation strategies and utilized genes of protective or transporter functions, resulting in “only-one-action” modifications. However, the limited success of this strategy might be due to the complexity of responses to abiotic stress, which generally involve the concerted action of many genes (polygenic) and therefore, the use of only one of them resulted in a minor effect (Fernandez-Muñoz 2005, Tuberosa and Salvi, 2006). An alternative recently development in different plant breeding programs is based on the utilization of genes coding for proteins with regulatory function (Yamaguchi-Shinozaki and Shinozaki 2006, Chinnusamy et al. 2004). The use of such genes has the main advantage to regulate simultaneously the action of many target genes involved in stress resistance, and therefore promotes greater effectiveness in the development of tolerance. The availability of an increasing number of plant genome sequences and new bioinformatics tools have allowed the identification many transcription factors, whose expression levels change in response to various abiotic stresses, including nutrient deficiencies (Riechmann et al. 2000; Chen et al. 2002). Most of these transcription factors belong to large families such as the bZIP, AP2/ERF, MYC, NAC, HSF, DOF and WRKY (Qu and Zhu 2006; Yamaguchi-Shinozaki and Shinozaki 2006), suggesting that salinity, dehydration and extreme temperatures mediate different mechanisms of transcriptional regulation of the stress response. However, the drawbacks of the pleiotropic effects of the transcription factors is that their overexpression might result in negative agronomic effects

Therefore, it is not obvious that all gene belonging to one of the large families of transcription factors has both a tolerance effect to nutrients deficiency and no negative effect on an agronomic trait like yield.

The use of a forward-genetic screening allowed the identification of a group of 12 transcription factors that when overexpressed in planta promote an enhancement of their growth and tolerance to nitrogen and sulphur deficiency, and carbon/nitrogen imbalance. The identified genes are also tested in field trials for yield performance in different stressed conditions. Therefore, the identified transcription factors can be used as new tools for improving tolerance under stress conditions. The present invention discloses also transgenic plants that overexpress transcription factors.

### SUMMARY OF THE INVENTION

In one aspect, the invention provides an expression cassette comprising at least one polynucleotide comprising a nucleotide sequence selected from the group consisting of:
a) a nucleotide sequence that encodes a polypeptide having the amino acid sequence set forth in SEQ ID NOs. 13-24; and
b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24.

This expression cassette can be used in vivo to increase the tolerance to abiotic stress, such as e.g., sulphur deficiency, nitrogen deficiency, or carbon/nitrogen imbalance.

In another aspect, the invention thus relates to a transgenic plant cell comprising the expression cassette described above.

In another aspect, the invention provides a transgenic plant comprising the transgenic plant cell described above. The invention also relates to a method of obtaining said transgenic plant comprising transforming said expression cassette into a plant. In one embodiment, the plant is a monocot. In another embodiment, the plant is a dicot.

In another aspect, the invention relates to a method for increasing or maintaining plant yield under stress conditions.

In agriculture, yield is the amount of product harvested from a given acreage (eg weight of seeds per unit area). It is often expressed in metric quintals (1 q=100 kg) per hectare in the case of cereals. It is becoming increasingly important to improve the yield of seed crops and one strategy to increase the yield is to increase the seed size, provided that there is not a concomitant decrease in seed number.

One important issue to be achieved in transgenic crop is obtaining plants capable of maintaining or increasing yield under stress conditions compared to normal conditions. Stress conditions can correspond for example to abiotic stress like light stress, extreme temperatures (heat, cold and freezing), drought (lack of precipitations) or soil contamination by salt. All these environmental stresses can more or less impair plant development growth and ultimately yield.

More specifically, the invention relates to a method for increasing or maintaining plant yield under non-optimal conditions for sulphur, nitrogen, and/or C/N balance, said method comprising a step of growing a transgenic plant as described above under conditions of non-optimal conditions for sulphur, nitrogen, and/or C/N balance, wherein:
the yield obtained from said transgenic plant grown under said non-optimal conditions is increased as compared to the yield obtained from a plant not containing the expression cassette of the invention, grown under said non-optimal conditions, or
the yield obtained from said transgenic plant grown under said non-optimal conditions is maintained as compared to the yield obtained from a transgenic plant and grown in optimal conditions for sulphur, nitrogen, and/or C/N balance

In another aspect, the invention provides a method for increasing or maintaining plant yield under optimal conditions for sulphur, nitrogen, and/or C/N balance, said method comprising a step of growing a transgenic plant under conditions of optimal conditions for sulphur, nitrogen, and/or C/N balance, wherein the yield obtained from said grown transgenic plant is increased as compared to the yield obtained from a plant not containing the expression cassette, grown under said optimal conditions

In another aspect, the invention also relates to a method for selecting a plant that can be used in a breeding process for obtaining a plant with improved yield under optimal or non-optimal conditions for for sulphur, nitrogen, and/or C/N balance. Said method comprises the step of selecting, in a population of plant, the plants containing the recombinant expression cassette of the invention.

In yet another aspect, the invention provides a method for identifying a plant with improved yield under optimal or under non optimal conditions for sulphur, nitrogen, and/or C/N balance comprising the step of identifying, in a population of plant, the plants containing the recombinant expression cassette of the invention.

### FIGURE LEGENDS

FIG. 1. Screening conditions used to identify TFs that enable plant growth under low nitrogen conditions.

(A) Phenotype of control (wt) plants grown on normal and low nitrogen medium. Representative pictures of 5 day-old control plants (Col) grown under standard (left) or low nitrogen conditions (right). Plants were grown on Petri dishes with MS 0,5X without nitrogen (Ref. M531, Phytotechnology Lab.) supplied with 30 mM N (15 mM KNO₃: 15 mM NH₄NO₃) and 1% sucrose as control medium, and MS 0,5X without nitrogen supplied with 0.1 mM N (0.05 mM KNO₃: 0.05 mM NH₄NO₃), 1 mM KCl (to compensate lower K⁺ concentration) and 1% sucrose as low N treatment. In both cases, media were supplemented with 0.7% (w/v) plant agar (Duchefa) and 0.05% (w/v) MES. After autoclaving, we added 1 ml Gamborg vitamins (Phytotechnology Lab; 1000× stock) and 1 ml ß-estradiol (SIGMA; 10 mM stock) per litre.

(B) Example of one of the transgenic lines identified in the screening. Representative pictures of wild type (left) and MYB overexpressing plants (TF) (right) grown under low nitrogen conditions.

FIG. 2. Screening conditions used to identify TFs enabling plant growth under C/N imbalance conditions.

(A) Phenotype of wild-type plants grown under control and C/N imbalance conditions. Representative pictures of 7-days old (Col) plants grown under control (left) or imbalance C/N conditions (right). Plants were grown on Petri dishes in MS 0,5X without nitrogen supplied with 30 mM N (15 mM KNO₃: 15 mM NH₄NO₃) and 100 mM Glucose as control medium, and 0.1 mM N (0.05 mM KNO₃: 0.05 mM NH₄NO₃), 1 mM KCl and 300 mM Glucose as C/N imbalance treatment. Agar, MES, vitamins and ß-estradiol were added as were described for low nitrogen screening.

(B) Example of one of the transgenic lines identified in the screening. Representative pictures of 7-days old wild type (left) and WRKY overexpressing plants (TF) (right) plants grown under C/N imbalance conditions.

FIG. 3. Conditions used in the screening to identify TFs that allow plant growth under sulphur-minus conditions.

(A) Phenotype of control plants growing under control and sulphur-minus medium. Representative pictures of 9-days old wild-type plants (Col) grown under control (left) or sulphur-minus conditions (right). Petri dishes containing control conditions (MS 0,5X,_2.5 mM KH₂PO₄, 2 mM MgS0₄, 1% sucrose) or S minus medium (MS 0,5X, 2.5 mM KH₂PO₄, 2 mM MgCl₂, 1% sucrose), both supplemented with 10 μM ß-estradiol. The pH was adjusted to 5.8 and 8.0 g/l of agarose was added before autoclaving. (Sulphur-containing and sulphur-free media composition is detailed in Table 2).

(B) Example of one of the transgenic lines identified in the screening. Representative pictures of 9-day old wild-type (left) and WRKY overexpressing transgenic plants (TF) (right) grown under sulphur minus conditions.

### DETAILED DESCRIPTION

The present invention relates to the identification of transcription factors conferring pleiotropic resistance to plants against various abiotic stresses. The present inventors have used a genetic screen to isolate transcription factors whose overexpression enables plant growth and development even under conditions of nutrient limitation. In particular, the present inventors have identified and characterized transcription factors which promote growth enhancement and tolerance to nitrogen and sulphur deficiency, and carbon/nitrogen imbalance, when said transcription factors are overexpressed.

The transcription factors of the invention are thus particularly useful because overexpression thereof confers increased tolerance to abiotic stress, such as sulphur deficiency, nitrogen deficiency, and/or carbon/nitrogen imbalance. This increased tolerance translates into an increased yield, both under stress and non-stress conditions.

The term “abiotic stress” is used herein in its regular meaning as the negative impact of non-living factors (e.g., drought, salinity, heat, cold, nutrient availability, metabolic balances) on a plant in a specific environment. Any of these abiotic stresses can delay growth and development, reduce productivity, and in extreme cases, cause the plant to die. Abiotic stress is thus a major limiting factor of plant growth and productivity, reducing average yields for most major crop plants by more than 50% (Wang et al., Planta (2003) 218: 1-14). It is indeed widely acknowledged that the ability to improve plant tolerance to abiotic stress would be of great economic advantage to farmers since it would allow for the cultivation of crops during adverse conditions and in territories where cultivation of crops may not otherwise be possible.

The term “sulphur deficiency” as used herein refers to sulphur deficiency that results in sulphur starvation of a plant when grown under sulphur deficient conditions. Thus, the expressions “increasing the resistance to sulphur deficiency” or “increased tolerance to sulphur deficiency” refer to the ability of the transcription factors disclosed herein to promote plant growth and development under conditions of sulphur deficiency, when said transcription factors are overexpressed.

Likewise, “nitrogen deficiency” as used herein refers to nitrogen deficiency that results in nitrogen starvation of a plant when grown under nitrogen deficient conditions, whereas the expressions “increasing the resistance to nitrogen deficiency” or “increased tolerance to nitrogen deficiency” refer to the ability of the transcription factors contemplated herein to promote plant growth and development under conditions of nitrogen deficiency, when said transcription factors are overexpressed.

In field trials evaluation two nitrogen conditions are generally used:
A normal (optimal) growing condition with an optimal Nitrogen fertilization. The applied Nitrogen rate is calculated using local guideline.
A nitrogen stress condition, where the applied Nitrogen rate is between 0 and 50% of the optimal Nitrogen rate.

After harvest, the stress intensity of the N stress condition can be characterized, based on the control seed yield lost compared to the control seed yield under the normal condition. Control seeds are generally non transgenic null segregant seeds.

The N stress intensity is generally characterized based on the following approximate categories.

| | Seed yield lost compare |
|---|---|
| Nitrogen stress level | to the optimal condition |
| Low N stress condition | 0 to 15% |
| Moderate N stress condition | 15% to 30% |
| Strong N stress condition | Above 30% |

The terms “carbon/nitrogen imbalance” or “C/N imbalance” refer to an alteration of the coordination between cellular carbon and nitrogen metabolisms. This coordination is crucial for sustaining optimal growth and development. For example, post-germinative growth is severely inhibited when the levels of carbon are high while nitrogen is limited. Thus, the expressions “increasing the resistance to carbon/nitrogen imbalance” or “increased tolerance to carbon/nitrogen imbalance” refer to the ability of the transcription factors contemplated herein to promote plant growth and development under conditions of C/N imbalance, when said transcription factors are overexpressed.

The invention thus relates to an isolated polynucleotide comprising a nucleotide sequence selected from the group consisting of: (a) a nucleotide sequence that encodes a polypeptide having the amino acid sequence set forth in SEQ ID NOs. 13-24; and (b) a nucleotide sequence encoding a homolog of any one of the polypeptides having the amino acid sequence set forth in SEQ ID NOs. 13-24. Preferably, said nucleic sequence is a nucleotide sequence set forth in SEQ ID NOs. 1-12.

The invention also relates to a polypeptide having the amino acid sequence set forth in SEQ ID NOs. 13-24, said polypeptide being a transcription factor which improves tolerance to stress derived from nutrient limitations. Preferably, the transcription factor of the invention improves tolerance to stress derived from nitrogen or sulphur deficiency or carbon/nitrogen imbalance when said transcription factor is overexpressed. More preferably, overexpression of the transcription factor of the invention results in an increase of the yield under both stress and non-stress conditions.

The term “overexpression” as used herein, refers to the increased expression of a polynucleotide encoding a protein. The term “expression”, as used herein, refers to the transcription and stable accumulation of sense (mRNA) or antisense RNA. Expression also includes translation of mRNA into a polypeptide. The term “increased” as used in certain embodiments means having a greater quantity, for example a quantity only slightly greater than the original quantity, or for example a quantity in large excess compared to the original quantity, and including all quantities in between. Alternatively, “increased” may refer to a quantity or activity that is at least 1%, 2%, 3%, 4%, 5%, 6%, 7%, 8%, 9%, 10%, 11%, 12%, 13%, 14%, 15%, 16%, 17%, 18%, 19% or at least 20% more than the quantity or activity for which the increased quantity or activity is being compared. The terms “increased”, “greater than”, and “improved” are used interchangeably herein.

As used herein, the term “transcription factor” refers to a protein that modulates gene expression by interaction with the transcriptional regulatory element (also called cis-motif) and cellular components for transcription, including RNA polymerase, Transcription Associated Factors (TAFs), chromatin-remodeling proteins, and any other relevant protein that impacts gene transcription. Preferably, the term “transcription factor” refers to trans-acting regulatory proteins which can bind to cis-acting elements, also called cis-acting motifs or cis-motifs, which are short DNA sequences located upstream of genes, or within introns. A transcription factor according to the invention may comprise one or more DNA-binding domains which mediate binding of the said transcription factor to said cis-acting sequences. Such DNA-binding domains are well known in the art and are used to classify transcription factors. They can be reliably used to identify transcription factors in putative proteins included by open reading frames identified in genomic databases (see e.g., Riechmann et al. 2000; Jin et al 2014; Perez-Rodriguez et al 2009; Naika et al 2013)

Plant genomes contain a great number of transcription factors, either demonstrated or only predicted. For example, the Arabidopsis thaliana genome codes for at least 1533 transcriptional regulators, accounting for ˜5.9% of its estimated total number of genes (Riechmann et al. (2000) Science 290: 2105-2109). The Database of Rice Transcription Factors (DRTF) is a collection of known and predicted transcription factors of Oryza sativa L ssp. indica and Oryza sativa L. ssp. japonica, and currently contains 2,025 putative transcription factors (TF) gene models in indica and 2,384 in japonica, distributed in 63 families (Gao et al. (2006) Bioinformatics 2006, 22(10): 1286-7).

The plant transcription factors are derived, e.g., from Arabidopsis thaliana and can belong, e.g., to one or more of the following transcription factor families: the AP2 (APETALA2) domain transcription factor family (Riechmann and Meyerowitz (1998) J. Biol. Chem. 379:633-646); the MYB transcription factor family (Martin and Paz-Ares (1997) Trends Genet. 13:67-73); the MADS domain transcription factor family (Riechmann and Meyerowitz (1997) J. Biol. Chem. 378:1079-1101); the WRKY protein family (Ishiguro and Nakamura (1994) Mol. Gen. Genet. 244:563-571); the ankyrin-repeat protein family (Zhang et al. (1992) Plant Cell 4:1575-1588); the miscellaneous protein (MISC) family (Kim et al. (1997) Plant J. 11:1237-1251); the zinc finger protein (Z) family (Klug and Schwabe (1995) FASEB J. 9: 597-604); the homeobox (HB) protein family (Duboule (1994) Guidebook to the Homeobox Genes, Oxford University Press); the CAAT-element binding proteins (Forsburg and Guarente (1989) Genes Dev. 3:1166-1178); the squamosa promoter binding proteins (SPB) (Klein et al. (1996) Mol. Gen. Genet. 1996 250:7-16); the NAM protein family; the IAA/AUX proteins (Rouse et al. (1998) Science 279:1371-1373); the HLH/MYC protein family (Littlewood et al. (1994) Prot. Profile 1:639-709); the DNA-binding protein (DBP) family (Tucker et al. (1994) EMBO J. 13:2994-3002); the bZIP family of transcription factors (Foster et al. (1994) FASEB J. 8:192-200); the BPF-1 protein (Box P-binding factor) family (da Costa e Silva et al. (1993) Plant J. 4:125-135); and the golden protein (GLD) family (Hall et al. (1998) Plant Cell 10:925-936).

The present invention is not limited to the transcription factors of SEQ ID NOs. 13-24 and the genes encoding said transcription factors, but is meant to encompass homologs thereof, including orthologues and paralogs.

A “homolog” as used herein is a gene related to a second gene by descent from a common ancestral DNA sequence. The term “homolog” may apply to the relationship between genes separated by speciation (orthologue), or to the relationship between genes originating via genetic duplication (see paralog).

As used herein, “orthologues” are genes in different species that evolved from a common ancestral gene by speciation. In contrast, “paralog” refers to homologs in the same species that evolved by genetic duplication of a common ancestral gene. Normally, orthologues retain the same function in the course of evolution. Preferably, the term “orthologue” or “orthologous” in reference to proteins means that said orthologous proteins are believed to be under similar regulation, have the same function and usually the same specificity in close organisms. Within the context of the present invention, the term “orthologue thereof” designates a related gene or protein from a distinct species, having a high degree of sequence similarity, and more particularly a level of sequence identity to the transcription factor of the invention of at least 50%, preferably at least 55%, 60%, 65%, 70%, 75%, 80%, 85%, especially preferably at least 90%, 95%, 97%, 98%, 99%, or more, for the coding sequence or amino acid sequences, respectively, and a transcription factor-like activity. An orthologue of such a transcription factor is most preferably a gene or protein from a distinct species having a common ancestor with said transcription factor, which is capable of modulating the transcription of plant gene, thus resulting in improved resistance to any one of the afore-mentioned stresses, and having a degree of sequence identity with said transcription factor of at least 50%, preferably at least 55%, 60%, 65%, 70%, 75%, 80%, 85%, especially preferably at least 90%, 95%, 97%, 98%, 99%, or more, for coding sequences. A nucleotide sequence of an orthologue in one species can be used to isolate the nucleotide sequence of the orthologue in another species (for example, maize, soybean, sunflower, sorghum, canola, wheat, alfalfa, cotton, rice, barley, millet, peanut, sugarcane or cocoa) using standard molecular biology and bioinformatics techniques. This can be accomplished, for example, using standard techniques in the art such as low-stringency hybridization or amplification of conserved sequences. All these techniques are well known to the person of skills in the art and need thus not be detailed here. In addition, homologs also encompass variants of the polynucleotides described above which are thus included within the scope of the invention. The term “variant”, as used herein, refers to polynucleotides or polypeptides that differ from the presently disclosed polynucleotides or polypeptides, respectively, in sequence from each other, and as set forth below.

Preferred orthologues according to the invention encompass the Zea mays polypeptides of SEQ ID NOs. 42-53 and the respective orthologues thereof listed in Table 3. Said homologs, including the polypeptides of SEQ ID NOs. 42-53, retain the same function as the polypeptides of sequences SEQ ID NOs. 13-24 respectively, and can be used for said polypeptides of SEQ ID NOs. 13-24. Even more preferably, said orthologues correspond to the Zea mays polypeptide of SEQ ID NOs. 42, 44, 50, and 53, and their homologues represented by the sequences of SEQ ID NOs. 54, 56, 62, 65, 66, 68, 74, and 77 as listed in table 3.

In particular, “polypeptide variants” refer to polypeptide sequences that are paralogs and orthologues of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may also confer an advantageous property such as a lack of immunogenicity. Thus, it will be readily appreciated by those of skill in the art, that any of a variety of polynucleotide sequences is capable of encoding the polypeptides and homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as a significant amount of the functional or biological activity of the polypeptide is retained. Commonly-used computer programs, including commercially available software such as e.g. Vector NTi (Life Technologies), can be used for determining adequate possible amino acid substitutions. These differences may produce silent changes and result in a functionally equivalent polypeptide.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide). Variant nucleotide sequences may encode different amino acid sequences, in which case such nucleotide differences will result in amino acid substitutions, additions, deletions, insertions, truncations or fusions with respect to the similar disclosed polynucleotide sequences. These variations may result in polynucleotide variants encoding polypeptides that share at least one functional characteristic, i.e. tolerance to at least one of the previously mentioned abiotic stresses. The degeneracy of the genetic code also dictates that many different variant polynucleotides can encode identical and/or substantially similar polypeptides in addition to the sequences represented by SEQ ID NOs. 1-12. Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent transcription factor.

Thus, it will be readily appreciated by those of skill in the art, that any of a variety of polynucleotide sequences is capable of encoding the transcription factors of the invention.

The nucleic acid molecules of the invention can be “optimized” for enhanced expression in plants of interest (see, for example, WO 91/16432; Perlak 1991; Murray 1989). In this manner, the open reading frames in genes or gene fragments can be synthesized utilizing plant-preferred codons (see, for example, Campbell [symbol] Gowri, 1990 for a discussion of host-preferred codon usage). Thus, the nucleotide sequences can be optimized for expression in any plant. It is recognized that all or any part of the gene sequence may be optimized. That is, partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shutting are known in the art (see, for example, Stemmer 1994; Stemmer 1994; Crameri 1997; Moore 1997; Zhang 1997; Crameri 1998; and U.S. Pat. Nos. 5,605,793 and 5,837,458).

Preferred homologs of any one of the transcription factors of the invention have a nucleic acid sequence of at least 50%, preferably at least 55%, 60%, 65%, 70%, 75%, 80%, 85%, especially preferably at least 90%, 95%, 97%, 98%, 99%, or more of sequence identity to a sequence selected in the group comprising SEQ ID NOs 1-12. More preferably, overexpression of any one of said homologs leads to increased resistance to at least one of sulphur deficiency, nitrogen deficiency and/or C/N imbalance.

According to a preferred embodiment of the present invention, preferred homologs of any one of the transcription factors of the invention have a nucleic acid sequence of at least 50%, preferably at least 55%, 60%, 65%, 70%, 75%, 80%, 85%, especially preferably at least 90%, 95%, 97%, 98%, 99%, or more of sequence identity to a sequence selected in the group comprising SEQ ID NOs 1-12, by conducting a global optimal alignment over the whole length of the respective sequences SEQ ID NOs 1-12, for example by using the algorithm of Needleman and Wunsch (J. Mol. Biol, 48(3): 443-453, 1972). More preferably, overexpression of any one of said homologs leads to increased resistance to at least one of sulphur deficiency, nitrogen deficiency and/or C/N imbalance.

Preferred homologues of the transcription factors of SEQ ID NOs. 16, 18 and 21 according to the invention include respectively the polypeptides of sequences SEQ ID NOs 25-27. Said preferred homologues are particularly advantageous since they lack any feature predicted to confer immunogenicity in the host plant.

Thus, in a preferred embodiment, the polynucleotide of the invention comprises a nucleotide sequence selected from the group consisting of: (a) a nucleotide sequence that encodes a polypeptide having the amino acid sequence set forth in SEQ ID NOs. 13-24; and (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs 13-24. Preferably, said polynucleotide comprises a nucleotide sequence set forth in SEQ ID NOs. 1-12.

In a more preferred embodiment, the polynucleotide of the invention comprises a nucleotide sequence selected from the group consisting of: (a) a nucleotide sequence that encodes a polypeptide having the amino acid sequence set forth in SEQ ID NOs. 13-24; and (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs 13-24 by conducting a global optimal alignment over the whole length of the respective sequences SEQ ID NOs 13-24, for example by using the algorithm of Needleman and Wunsch (J. Mol. Biol, 48(3): 443-453, 1972).

In a further preferred embodiment, the nucleic acid sequence of the polynucleotide of the invention is selected from the group consisting of: (a) a nucleotide sequence that encodes a polypeptide having the amino acid sequence set forth in SEQ ID NOs. 13-24; and (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24, and encoding a transcription factor conferring resistance to at least one of sulphur deficiency, nitrogen deficiency, and C/N imbalance, when said transcription factor is overexpressed. Preferably, said polynucleotide comprises a a nucleotide sequence set forth in SEQ ID NOs. 1-12.

In another further preferred embodiment, the nucleic acid sequence of the polynucleotide of the invention is selected from the group consisting of: (a) a nucleotide sequence that encodes a polypeptide having the amino acid sequence set forth in SEQ ID NOs. 13-24; and (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24, by conducting a global optimal alignment over the whole length of the respective sequences SEQ ID NOs 13-24, for example by using the algorithm of Needleman and Wunsch (J. Mol. Biol, 48(3): 443-453, 1972), and encoding a transcription factor conferring resistance to at least one of sulphur deficiency, nitrogen deficiency, and C/N imbalance, when said transcription factor is overexpressed. Preferably, said polynucleotide comprises a nucleotide sequence set forth in SEQ ID NOs. 1-12.

The term “sequence identity” refers to the identity between two peptides or between two nucleic acids. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences. Since two polypeptides may each (i) comprise a sequence (i.e. a portion of a complete polynucleotide sequence) that is similar between two polynucleotides, and (ii) may further comprise a sequence that is divergent between two polynucleotides, sequence identity comparisons between two or more polynucleotides over a “comparison window” refers to the conceptual segment of at least 20 contiguous nucleotide positions wherein a polynucleotide sequence may be compared to a reference nucleotide sequence of at least 20 contiguous nucleotides and wherein the portion of the polynucleotide sequence in the comparison window may comprise additions or deletions (i.e. gaps) of 20 percent or less compared to the reference sequence (which does not comprise additions or deletions) for optimal alignment of the two sequences.

To determine the percent identity of two amino acids sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position. The percent identity between the two sequences is a function of the number of identical positions shared by the sequences. Hence % identity=number of identical positions/total number of overlapping positions×100.

In this comparison the sequences can be of the same length or can be different in length. Optimal alignment of sequences for determining a comparison window may be conducted by the local homology algorithm of Smith and Waterman (J. Theor. Biol., 91(2): 370-380, 1981), by the homology alignment algorithm of Needleman and Wunsch (J. Mol. Biol, 48(3): 443-453, 1972), by the search for similarity via the method of Pearson and Lipman (Proc. Natl. Acad. Sci. U.S.A., 85(5): 2444-2448, 1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection.

One preferred strategy used to identify orthologues between species is the BBMH approaches (Best Blast Mutual Hits). This method allows to have the best orthologous sequences between two species. Each protein from specie A is blasted against all proteins from specie B. The best match from specie B to the initial query A protein should then have that same A protein as its best match in the reciprocal search. The best alignment (i.e. resulting in the highest percentage of identity over the comparison window) generated by the various methods is selected BLASTP program (especially the BLASTP 2.2.29 program) (Altschul et al, (1997), Nucleic Acids Res. 25:3389-3402; Altschul et al, (2005) FEBS J. 272:5101-5109) can also be used with the following algorithm parameters:
Expected threshold: 10
Word size: 3
Max matches in a query range: 0
Matrix: BLOSUM62
Gap Costs: Existence 11, Extension 1.
Compositional adjustments: Conditional compositional score matrix adjustment
No filter for low complexity regions
The proteins that can be used in the context of the above construct are preferably the ones that present a Max score above 1000.

The term “sequence identity” means that two polynucleotide or polypeptide sequences are identical (i.e. on a nucleotide by nucleotide or an amino acid by amino acid basis) over the window of comparison. The term “percentage of sequence identity” is calculated by comparing two optimally aligned sequences over the window of comparison, determining the number of positions at which the identical nucleic acid base (e.g. A, T, C, G or U) occurs in both sequences to yield the number of matched positions, dividing the number of matched positions by the total number of positions in the window of comparison (i.e. the window size) and multiplying the result by 100 to yield the percentage of sequence identity. The same process can be applied to polypeptide sequences. The percentage of sequence identity of a nucleic acid sequence or an amino acid sequence can also be calculated using BLAST software (Version 2.06 of September 1998) with the default or user defined parameter.

According to a preferred embodiment of the present invention, the “sequence identity” is calculated after conducting a global optimal alignment over the whole length of the sequence according to the invention, for example by using the algorithm of Needleman and Wunsch (J. Mol. Biol, 48(3): 443-453, 1972).

The term “sequence similarity” means that amino acids can be modified while retaining the same function. It is known that amino acids are classified according to the nature of their side groups and some amino acids such as the basic amino acids can be interchanged for one another while their basic function is maintained.

In order to engineer plants with desired enhanced resistance to said abiotic stress conditions, one skilled in the art can introduce transcription factors or nucleic acids encoding transcription factors into the plants. For example, one of skill in the art can prepare an expression cassette or expression vector that can express one or more encoded transcription factors, wherein regulatory sequences, such as e.g., promoter sequences and/or transcription termination sequences, are operably linked to the coding region of interest. Plant cells can be transformed by the expression cassette or an expression vector comprising said cassette, and whole plants (and their seeds) can be generated from the plant cells that were successfully transformed with the promoter and/or transcription factor nucleic acids.

Therefore, in another aspect, the invention provides an expression cassette comprising at least one polynucleotide as described above, i.e., an expression cassette comprising at least one polynucleotide comprising a nucleotide sequence selected from the group consisting of: (a) a nucleotide sequence that encodes a polypeptide having the amino acid sequence set forth in SEQ ID NOs. 13-24; and (b) a nucleotide sequence that encodes a homolog of any one of the transcription factors having the amino acid sequence set forth in SEQ ID NOs. 13-24. Preferably, said polynucleotide comprises a a nucleotide sequence set forth in SEQ ID NOs. 1-12.

Preferably, the expression cassette of the invention comprises at least one polynucleotide as described above, i.e., an expression cassette comprising at least one polynucleotide comprising a nucleotide sequence selected from the group consisting of: (a) a nucleotide sequence that encodes a polypeptide having the amino acid sequence set forth in SEQ ID NOs. 13-24; and (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24. Preferably, said expression cassette comprises a polynucleotide comprises a polynucleotide comprising a nucleotide sequence set forth in SEQ ID NOs. 1-12.

More preferably, the expression cassette of the invention comprises at least one polynucleotide as described above, i.e., an expression cassette comprising at least one polynucleotide comprising a nucleotide sequence selected from the group consisting of: (a) a nucleotide sequence that encodes a polypeptide having the amino acid sequence set forth in SEQ ID NOs. 13-24; and (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24, by conducting a global optimal alignment over the whole length of the respective sequences SEQ ID NOs 13-24, for example by using the algorithm of Needleman and Wunsch (J. Mol. Biol, 48(3): 443-453, 1972).

Also preferably, the expression cassette of the invention comprises at least one polynucleotide as described above, i.e., an expression cassette comprising at least one polynucleotide comprising a nucleotide sequence selected from the group consisting of: (a) a nucleotide sequence that encodes a polypeptide having the amino acid sequence set forth in SEQ ID NOs. 13-24; (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24, said polypeptide being a transcription factor conferring resistance to at least one of sulphur deficiency, nitrogen deficiency, and/or C/N imbalance, when said transcription factor is overexpressed. Preferably, said expression cassette comprises a polynucleotide comprises a polynucleotide comprising a nucleotide sequence set forth in SEQ ID NOs. 1-12.

And also more preferably, the expression cassette of the invention comprises at least one polynucleotide as described above, i.e., an expression cassette comprising at least one polynucleotide comprising a nucleotide sequence selected from the group consisting of: (a) a nucleotide sequence that encodes a polypeptide having the amino acid sequence set forth in SEQ ID NOs. 13-24; (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24, by conducting a global optimal alignment over the whole length of the respective sequences SEQ ID NOs 13-24, for example by using the algorithm of Needleman and Wunsch (J. Mol. Biol, 48(3): 443-453, 1972), said polypeptide being a transcription factor conferring resistance to at least one of sulphur deficiency, nitrogen deficiency, and/or C/N imbalance, when said transcription factor is overexpressed.

Even more preferably, the expression cassette of the invention has a nucleotide sequence selected from the sequences represented by SEQ ID NOs. 30-41.

The term “expression cassette” as used herein refers to a DNA fragment comprising a polynucleotide of interest, e.g., a polynucleotide encoding one of the transcription factors of the invention, which is operably linked to various regulatory elements that regulate the expression of the gene sequence, such as e.g., promoter sequences and enhancer sequences.

“Operably-linked” refers to the association of nucleic acid sequences on a single nucleic acid fragment so that the function of one is affected by the other. For example, a regulatory DNA sequence is said to be “operably linked to” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Preferably, the coding sequences of the invention are operably-linked to regulatory sequences in the sense orientation.

The term “regulatory sequence” or “regulatory element” as used herein refers to polynucleotide sequences which are necessary to affect the expression and processing of coding sequences to which they are ligated. Such regulatory sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. Examples of regulator sequences are described in e.g., Goeddel, Gene Expression Technology: Methods in Enzymology 185, Academic Press, San Diego, Calif. (1990) and Gruber and Crosby, in: Methods in Plant Molecular Biology and Biotechnology, eds. Click and Thompson, Chapter 7, 89-108, CRC Press: Boca Raton, Fla.

Preferably, the regulatory sequences of the invention comprise promoter sequences, i.e., the transcription factor-encoding polynucleotide of the invention is preferably operably linked to a promoter, which provides for expression of mRNA from the transcription factor nucleic acids. A transcription factor-encoding nucleic acid is operably linked to the promoter when it is located downstream from the promoter, to thereby form an expression cassette.

In a preferred embodiment, the expression cassette of the invention therefore comprises at least one polynucleotide of the invention as described above, wherein said polynucleotide is operably linked to a promoter. In a further preferred embodiment, the expression cassette of the invention has a sequence selected in the group consisting of SEQ ID NOs: 78-89.

As used herein, the term “promoter” refers to a nucleotide sequence, usually upstream (5′) to its coding sequence, which controls the expression of the coding sequence by providing the recognition for RNA polymerase and other factors required for proper transcription. A “promoter” as used herein includes a minimal promoter that is a short DNA sequence composed of a TATA box and other sequences that serve to specify the site of transcription initiation, to which regulatory elements are added for control of expression. A “promoter” also refers to a nucleotide sequence that includes a minimal promoter plus regulatory elements that is capable of controlling the expression of a coding sequence or functional RNA. For example, promoter sequences may contain regulatory sequences such as enhancer sequences that can influence the level of gene expression.

The promoter is typically a promoter functional in plants and/or seeds, and can be a promoter functional during plant growth and development. Promoters useful in the expression cassettes of the invention include any promoter that is capable of initiating transcription in a plant cell. Such promoters include, but are not limited to, those that can be obtained from plants, plant viruses, and bacteria that contain genes that are expressed in plants, such as Agrobacterium and Rhizobium and also synthetic promoters.

The promoter may be constitutive, inducible, developmental stage-preferred, cell type-preferred, tissue-preferred, or organ-preferred. Constitutive promoters are active under most conditions. Examples of constitutive promoters useful for expression include the pCRV promoter (Depigny-This et al., 1992, Plant Molecular Biology, 20: 467-479), the CsVMV promoter (Verdaguer et al., 1998, Plant Mol Biol. 6: 1129-39), the ubiquitin 1 promoter of maize (Christensen et al., 1996, Transgenic. Res., 5: 213).

Other examples of constitutive promoters include the CaMV 19S and 35S promoters (Odell et al., 1985, Nature 313: 810-812), the sX CaMV 35S promoter (May et al., 1987, Science 236: 1299-1302) the Sep1 promoter, the rice actin promoter (McElroy et al., 1990, Plant Cell, 2:163-171)), the Arabidopsis actin promoter, the maize ubiquitin promoter (Christensen et al., 1989, Plant Molec. Biol, 18: 675-689), pEmu (Last et al., 1991, Theor. Appl. Genet, 81: 581-588), the figwort mosaic virus 35S promoter, the Smas promoter (Velten et al., 1984, EMBO J 3:2723-2730), the super-promoter (U.S. Pat. No. 5,955,646), the GRP1-8 promoter, the cinnamyl alcohol dehydrogenase promoter (U.S. Pat. No. 5,683,439), promoters from the T-DNA of Agrobacterium, such as manopine synthase, nopaline synthase, and octopine synthase, the small subunit of ribulose biphosphate carboxylase (ssuRUBISCO) promoter, and the like. A preferred constitutive promoter according to the invention is the rice actin promoter (McElroy et al., 1990), and more preferably the rice actin promoter linked to the rice actin intron which is represented by the sequence SEQ ID NO: 28.

Plant gene expression can also be facilitated via an inducible promoter (For review, see Gatz, 1997, Annu. Rev. Plant Physiol. Plant Mol. Biol. 48: 89-108). Inducible promoters are preferentially active under certain environmental conditions, such as the presence or absence of a nutrient or metabolite, heat or cold, light, pathogen attack, anaerobic conditions, and the like. For example, the hsp80 promoter from Brassica is induced by heat shock; the PPDK promoter from maize is induced by light (Matsuoka et al, 1993); the PR-1 promoters from tobacco, Arabidopsis, and maize are inducible by infection with a pathogen; and the Adh1 promoter from Arabidopsis is induced by hypoxia and cold stress. Chemically-inducible promoters are especially suitable if gene expression is wanted to occur in a time-specific manner. Examples of such promoters are a salicylic acid-inducible promoter (WO 95/19443), a tetracycline-inducible promoter (Gatz et al., 1992, Plant J. 2: 397-404), an ethanol-inducible promoter (WO 93/21334), and a ß-estradiol-inducible promoter (Zuo et al., Plant J, 24: 265-273, 2000).

Stress-inducible promoters are preferentially active under one or more of the following stresses: sub-optimal conditions associated with salinity, drought, temperature, metal, chemical, pathogenic, and oxidative stresses. Stress-inducible promoters include, but are not limited to, Cor78 (Chak et al., 2000, Planta 210: 875-883; Hovath et al., 1993. Plant Physiol. 103: 1047-1053), Cor15a (Artus et al., 1996, Proc. Natl. Acad Sci. 93(23): 13404-09), Rci2A (Medina et al., 2001, Plant Physiol. 125: 1655-66; Nylander et al., 2001, Plant Mol. Biol. 45: 341-52; Navarre and Goffeau, 2000, EMBO J. 19: 2515-24; Capel et al., 1997, Plant Physiol. 115: 569-76), Rd22 (Xiong et al., 2001, Plant Cell 13: 2063-83; Abe et al., 1997, Plant Cell 9: 1859-68; Iwasaki et al., 1995, Mol. Gen. Genet. 247: 391-8), cDet6 (Lang and Palve, 1992, Plant Mol. Biol. 20: A51-62), ADH1 (Hoeren et al., 1998, Genetics 149: 479-90), KAT1 (Nakamura et al., 1995, Plant Physiol. 109: 371-4), KST1 (Müller-Röber et al., 1995, EMBO 14: 2409-16), Rha1 (Terryn et al., 1993, Plant Cell 5: 1761-9; Terryn et al., 1992, FEBS Lett. 299(3):287-90), ARSK1 (Atkinson et al., 1997, GenBank Accession # L22302, and WO 97/20057), PtxA (Plesch et al., GenBank Accession # X67427). SbHRGP3 (Ahn et al., 1996, Plant Cell 8: 1477-90), GH3 (Liu et al., 1994, Plant Cell 6: 645-57), the pathogen inducible PRP1-gene promoter (Ward et al., 1993, Plant. Mol. Biol. 22: 361-366), the heat inducible hsp80-promoter from tomato (U.S. Pat. No. 5,187,267), cold inducible alpha-amylase promoter from potato (WO 96/12814), or the wound-inducible pinII-promoter (EP 0 375 091). For other examples of drought, cold, and salt-inducible promoters, such as the RD29A promoter, see Yamaguchi-Shinozalki et al., 1993, Mol. Gen. Genet. 236: 331-340.

Developmental stage-preferred promoters are preferentially expressed at certain stages of development. Tissue- and organ-preferred promoters include those that are preferentially expressed in certain tissues or organs, such as leaves, roots, seeds, or xylem. Examples of tissue-preferred and organ-preferred promoters include, but are not limited to fruit-preferred, ovule-preferred, male tissue-preferred, seed-preferred, integument-preferred, ear-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters, and the like. Seed-preferred promoters are preferentially expressed during seed development and/or germination, For example, seed preferred promoters can be embryo-preferred, endosperm-preferred, or seed coat-preferred. See Thompson et al., 1989, BioEssays 10: 108, Examples of seed preferred promoters include, but are not limited to, cellulose synthase (ce1A), Cim1, gamma-zein, globulin-1, maize 19 kD zein (cZ19B1), and the like.

Other suitable tissue-preferred or organ-preferred promoters include the napin-gene promoter from rapeseed (U.S. Pat. No. 5,608,152), the USP-promoter from Vicia faba (Baeumlein et al., 1991, Mol. Gen. Genet. 225(3):459-67), the oleosin-promoter from Arabidopsis (WO 98/45461), the phaseolin-promoter from Phaseolus vulgaris (U.S. Pat. No. 5,504,200), the Bce4-promoter from Brassica (WO 91/139567), or the legumin B4 promoter (LeB4; Baeumlein et al., 1992, Plant Journal, 2(2):233-9), as well as promoters conferring seed specific expression in monocot plants like maize, barley, wheat, rye, rice, etc. Suitable promoters to note are the 1pt2 or 1pt1-gene promoter from barley (WO 95/15389 and WO 95/23230) or those described in WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the ß-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

In another preferred embodiment, the regulatory sequences of the invention comprise termination sequences. The term “terminator” or “termination sequence” generally refers to a 3′ flanking region of a gene that contains nucleotide sequences which regulate transcription termination and typically confer RNA stability. The use of recombinant terminator sequences is established in the art (Guerineau et al., (1991), Mol. Gen. Genet., 226: 141-144; Proudfoot, (1991), Cell, 64: 671-674; Sanfacon et al., (1991), Genes Dev., 5: 141-149; Mogen et al., (1990), Plant Cell, 2: 1261-1272; Munroe et al., (1990), Gene, 91: 151-158; Ballas et al., (1989), Nucleic Acids Res., 17: 7891-7903; Joshi et al., (1987), Nucleic Acid Res., 15: 9627-9639). Exemplary of such terminators include Agrobacterium tumefaciens nopaline synthase terminator (Tnos), Agrobacterium tumefaciens mannopine synthase terminator (Tmas), the CaMV 35S terminator (T35S), the pea ribulose bisphosphate carboxylase small subunit termination region (TrbcS), and the Tnos termination region. Particularly preferred transcription terminators according to the invention include the Arabidopsis thaliana Sac66 polyadenylation sequence of SEQ ID NO: 29 (Jenkins et al., 1999).

Techniques for operably linking a promoter and/or a transcription terminator to a nucleic acid sequence are known in the art. A transcription factor-encoding nucleic acid can be combined with a selected promoter by standard methods to yield an expression cassette, for example, as described in Sambrook et al. (Molecular Cloning: A Laboratory Manual. Third Edition (Cold Spring Harbor, N.Y.: Cold Spring Harbor Press (2000)). Briefly, a plasmid containing a plant promoter such as any of the promoters described above can be constructed as described in Jefferson (Plant Molecular Biology Reporter 5: 387-405 (1987)) or obtained from a commercial source. Typically, these plasmids are constructed to have multiple cloning sites having specificity for different restriction enzymes downstream from the promoter. Preferably, said plasmid contains transcription termination sequences located downstream of the multiple cloning site. The transcription factor nucleic acids can be subcloned downstream from the promoter using restriction enzymes and positioned to ensure that the transcription factor DNA is inserted in proper orientation with respect to the promoter and/or the transcription terminator so that the DNA can be expressed. Once the transcription factor nucleic acid is operably linked to a promoter and/or a transcription terminator, the expression cassette so formed can be subcloned into a plasmid or other vector (e.g., a destination vector).

In another aspect, the present invention provides a vector containing an expression cassette as disclosed above.

The term “vector”, as used herein, is intended to refer to a nucleic acid molecule capable of transporting another nucleic acid to which it has been linked. The vectors useful in the invention include, but are not limited to, plasmids, phagemids, viruses, other vehicles derived from viral or bacterial sources that have been manipulated by the insertion or incorporation of one or more nucleic acid sequences encoding transcription factors according to the invention. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced. Other vectors can be integrated into the genome of a host cell upon introduction into the host cell, and thereby are replicated along with the host genome.

More preferably, the vector of the invention is an expression vector. The term “expression vector” as used herein refers to a DNA sequence capable of directing expression of a particular nucleotide sequence to which they are operably linked in an appropriate host cell. Advantageously, the expression vector of the invention is functional in plants, i.e., is capable of directing transcription of the nucleic acid of interest in plants. More advantageously, the expression vector of the invention comprises an expression cassette as described above, i.e. a nucleotide sequence encoding a transcription factor according to the invention, operably linked to regulatory sequences such as e.g., promoter sequences and/or transcription terminator sequences.

For direct gene transfer techniques, where the polynucleotide or expression cassette is introduced directly into the plant cell, a simple bacterial cloning vector such as pUC19 is suitable. Alternatively, more complex vectors may be used in conjunction with Agrobacterium-mediated processes. Suitable vectors are derived from Agrobacterium tumefaciens or rhizogenes plasmids or incorporate essential elements from such plasmids. Agrobacterium vectors may be of co-integrate (EP 0 116 718) or binary type (EP 0 120 516).

Advantageously, the vector carrying the expression cassette of the invention also comprises a selection marker, in order to facilitate identification of the transformed plant cells. Selection markers include, but are not limited to, antibiotic resistance genes, herbicide resistance genes or visible genes. A number of selective agents and resistance genes are known in the art. (see, e.g., Hauptmann et al., 1988; Dekeyser et al., 1988; Eichholtz et al., 1987; and Meijer et al., 1991). Notably the selectable marker used can be the bar gene conferring resistance to bialaphos (White et al., 1990), the sulfonamide herbicide Asulam resistance gene, sul (described in WO 98/49316) encoding a type I dihydropterate synthase (DHPS), the nptπ gene conferring resistance to a group of antibiotics including kanamycin, G418, paromomycin and neomycin (Bevan et al., 1983), the hph gene conferring resistance to hygromycin (Gritz et al., 1983), the EPSPS gene conferring tolerance to glyphosate (U.S. Pat. No. 5,188,642), the HPPD gene conferring resistance to isoxazoles (WO 96/38567), the gene encoding for the GUS enzyme, the green fluorescent protein (GFP), expression of which, confers a recognizable physical characteristic to transformed cells, the chloramphenicol transferase gene, expression of which, detoxifies chloramphenicol. The choice and the use of a selection marker are well known to the person of skills in the art.

In another aspect, the invention provides a plant cell comprising an expression cassette or an expression vector as disclosed above.

As used herein, the term “plant cell” refers to a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ differentiated into a structure that is present at any stage of a plant's development. As used herein, a “plant cell” also means a plant cell that is transformed with stably-integrated, non-natural, recombinant DNA. Advantageously, said transformed plant cell (e.g., embryonic cells or other cell lines) can regenerate fertile transgenic plants and/or seeds.

In a preferred embodiment, a plant cell according to the invention is thus a transgenic plant cell. As used herein a “transgenic plant cell” means a plant cell comprising an expression cassette or an expression vector as disclosed above and enabling the overexpression of a transcription factor of the invention. A “transgenic plant” is a plant having one or more transgenic plant cells, i.e., one or more plant cells that contain an expression cassette as described above.

Thus, in another aspect, the present invention relates to a transgenic plant comprising an expression cassette or an expression vector as disclosed above. Advantageously, the overexpression of a transcription factor as described above is enabled in the transgenic plant of the invention.

A “plant” according to the invention refers to any plant, particularly to agronomically useful plants (e.g., seed plants). Preferably, the term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat), ear and fruits (the mature ovary), plant tissues (e.g. vascular tissue, ground tissue, and the like) and cells (e.g. guard cells, egg cells, trichomes and the like), and progeny of same.

Also included within the present invention are seeds of any of these transgenic plants.

Thus in another aspect, the invention also relates to a seed of a transgenic plant disclosed herein, i.e., a plant transformed with a cassette enabling the expression of a transcription factor conferring increased tolerance to at least one of sulphur deficiency, nitrogen deficiency and/or C/N imbalance.

The invention also relates to a plant obtained from the seed of said transgenic plant disclosed herein. In a preferred embodiment, said plant obtained from said seed overexpresses one transcription factor conferring increased tolerance to at least one of sulphur deficiency, nitrogen deficiency and/or C/N imbalance.

The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous. Included within the scope of the invention are all genera and species of higher and lower plants of the plant kingdom. Included are furthermore the mature plants, seed, shoots and seedlings, and parts, propagation material (for example seeds and fruit) and cultures, for example cell cultures, derived therefrom.

Annual, perennial, monocotyledonous and dicotyledonous plants are preferred host organisms for the generation of transgenic plants according to the invention. Most preferably, the plant which can be used in the method of the invention is dicot or a monocot.

Dicotyledonous plants which can be used in the invention include, for example, plants from the family of the Umbelliferae, particularly the genus Daucus (very particularly the species carota (carrot)) and Apium (very particularly the species graveolens var. dulce (celery)) and many others; the family of the Solanaceae, particularly the genus Lycopersicon, very particularly the species esculentum (tomato) and the genus Solanum, very particularly the species tuberosum (potato) and melongena (aubergine), tobacco and many others; and the genus Capsicum, very particularly the species annum (pepper) and many others; the family of the Malvaceae, particularly the genus Gossypium (cotton), very particularly the species arboretum; very particularly the species herbaceum, very particularly the species hirsutum, very particularly the species barbadense particularly the genus Theobroma, very particularly the species cacao (cacao tree or cocoa tree), and many others; the family of the Leguminosae, particularly the genus Glycine, very particularly the species max (soybean); and the genus Arachis, very particularly the species hypogaea (peanut), and many others; and the family of the Cruciferae, particularly the genus Brassica, very particularly the species napus (very particularly the species napus var. napus (canola)), campestris (beet), oleracea cv Tastie (cabbage), oleracea cv Snowball Y (cauliflower) and oleracea cv Emperor (broccoli); and the genus Arabidopsis, very particularly the species thaliana; and the genus Medicago, very particularly the species sativa (alfalfa); and the genus Pisum, very particularly the species sativum (pea); and many others; the family of the Asteraceae or Compositae, particularly the genus Lactuca, very particularly the species sativa (lettuce); and the genus Helianthus, very particularly the species annuus (sunflower), and many others.

The transgenic plants according to the invention may also be selected among monocotyledonous plants. The term “monocotyledonous plant” when referring to a transgenic plant according to the invention or to the source of the transcription regulating sequences of the invention is intended to comprise all genera, families and species of monocotyledonous plants. Preferred are Gramineae plants such as, for example, cereals such as maize, rice, wheat, barley, sorghum, millet, rye, triticale, or oats, and other non-cereal monocotyledonous plants such as sugarcane or banana. Especially preferred are corn (maize), rice, barley, wheat, rye, and oats. Most preferred are all varieties of the specie Zea mays and Oryza sativa.

In a preferred embodiment, the plant of the invention is a monocotyledonous plant, most preferably selected form the group consisting of Zea mays (corn), Oryza sativa (rice), Triticum aestivum (wheat), Hordeum vulgare (barley), and Avena sativa (oats).

In another preferred embodiment, said plant is selected from the group consisting of: maize, soybean, sunflower, sorghum, canola, wheat, alfalfa, cotton, rice, barley, millet, and sugar cane.

In another aspect, the invention provides a method for the production of a transgenic plant, said method comprising the transformation of a plant by a nucleic acid encoding a transcription factor as described above, e.g., an expression cassette or an expression vector.

In a preferred embodiment, the invention relates to a method for the production of a transgenic plant, said method comprising the step of transforming a plant with a nucleic acid encoding a transcription factor as described above, wherein said transgenic plant has increased tolerance to at least one of sulphur deficiency, nitrogen deficiency and/or C/N imbalance. In a further preferred embodiment, the method of the invention comprises the step of transforming said plant with an expression cassette as described above.

The terms “transformation” and “transforming” refer to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

Transformation of the cells of the plant tissue source can be conducted by any one of a number of methods known to those of skill in the art. Examples are: transformation by direct DNA transfer into plant cells by electroporation (U.S. Pat. No. 5,384,253 and U.S. Pat. No. 5,472,869, Dekeyser et al., The Plant Cell. 2: 591-602 (1990)); direct DNA transfer to plant cells by PEG precipitation (Hayashimoto et al., Plant Physiol. 93: 857-863 (1990)); direct DNA transfer to plant cells by microprojectile bombardment (McCabe et al., Bio/Technology. 6: 923-926 (1988); Gordon-Kamm et al., The Plant Cell. 2: 603-618 (1990); U.S. Pat. No. 5,489,520; U.S. Pat. No. 5,538,877; and U.S. Pat. No. 5,538,880) and DNA transfer to plant cells via infection with Agrobacterium.

Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf-disk protocol (Horsch et al., Science 227:1229-1231 (1985). Monocots such as Zea mays can be transformed via microprojectile bombardment of embryogenic callus tissue or immature embryos, or by electroporation following partial enzymatic degradation of the cell wall with a pectinase-containing enzyme (U.S. Pat. No. 5,384,253; and U.S. Pat. No. 5,472,869). For example, embryogenic cell lines derived from immature Zea mays embryos can be transformed by accelerated particle treatment as described by Gordon-Kamm et al. (The Plant Cell. 2: 603-618 (1990)) or U.S. Pat. No. 5,489,520; U.S. Pat. No. 5,538,877 and U.S. Pat. No. 5,538,880. Excised immature embryos can also be used as the target for transformation prior to tissue culture induction, selection and regeneration as described in WO 95/06128. Furthermore, methods for transformation of monocotyledonous plants utilizing Agrobacterium tumefaciens have been described in EP 0 604 662 and EP 0 672 752.

Methods such as microprojectile bombardment or electroporation are carried out with “naked” DNA where the expression cassette may be simply carried on any E. coli-derived plasmid cloning vector. In the case of viral vectors, it is desirable that the vectors retain replication functions, but not have functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are preferred Zea mays tissue sources. Selection of tissue sources for transformation of monocots is described in detail in WO 95/06128.

The transformation is carried out under conditions directed to the plant tissue of choice. The plant cells or tissue are exposed to the DNA or RNA carrying the transcription factor nucleic acids for an effective period of time. This may range from a less than one second pulse of electricity for electroporation to a 2-3 day co-cultivation in the presence of plasmid-bearing Agrobacterium cells. Buffers and media used will also vary with the plant tissue source and transformation protocol. Many transformation protocols employ feeder layer of suspended culture cells (tobacco or Black Mexican Sweet corn, for example) on the surface of solid media plates, separated by a sterile filter paper disk from the plant cells or tissues being transformed.

It is known that upon transformation of nucleic acids into plant cells, only a minority of said cells will take up the foreign DNA and, if desired, integrate it into their genome. This is dependent upon the technique of transformation, as well as on the vector used. In order to facilitate identification and selection of transformants, a selection marker is usually introduced along the expression cassette in the target plant cells, as detailed above. Generally after transformation, plant cells are selected for the presence of the selection marker, following which the transformed cell is regenerated into a whole plant. To select transformed plants, the plant material obtained in the transformation is subjected to conditions selecting for the presence of the selection marker. Transformed plants can thus be distinguished from untransformed plants. Whole plants can then be regenerated from transformed plant cells by culturing said cells on media that support regeneration of plants, followed by maturation of the transgenic plant.

Mature plants are obtained from cell lines that are known to express the desired trait. The term “trait” refers to a physiological, morphological, biochemical or physical characteristic of a plant or particular plant material or cell. In some instances, this characteristic is visible to the human eye, such as seed or plant size, or can be measured by available biochemical techniques, such as the protein, starch or oil content of seed or leaves or by the observation of the expression level of genes, e.g., by employing Northern analysis, RT-PCR, microarray gene expression assays or reporter gene expression systems, or by agricultural observations such as stress tolerance or yield. Preferably, the trait of the invention is detected by assessing tolerance to sulphur deficiency, nitrogen deficiency and/or C/N imbalance. Alternatively, the trait is detected by assessing yield under specific conditions.

Thus, in a preferred embodiment, the invention relates to a method for the production of a transgenic plant, said method, comprising the steps of:
a) transforming a plant cell with a nucleic acid encoding a transcription factor of the invention;
b) selecting a plant cell having increased tolerance to at least one of sulphur deficiency, nitrogen deficiency and/or C/N imbalance;
   wherein said transgenic plant has increased tolerance to at least one of sulphur deficiency, nitrogen deficiency and/or C/N imbalance

In another preferred embodiment, the method of the invention comprises the steps of:
a) transforming a plant with an expression cassette of the invention; and
b) selecting a plant cell having increased tolerance to at least one of sulphur deficiency, nitrogen deficiency and/or C/N imbalance.

The regenerated transformed plant can be propagated by a variety of means, such as clonal propagation or classical breeding techniques. In some embodiments, the regenerated plants are self-pollinated. In addition, pollen obtained from regenerated plants can be crossed to seed-grown plants of agronomically important inbred lines, i.e., true-breeding lines resulting from at least five successive generations of controlled self-fertilization or of backcrossing to a recurrent parent with selection or its equivalent. In some case, pollen from these agronomically important inbred lines is used to pollinate said regenerated plants. The trait, i.e., increased tolerance to abiotic stress such as sulphur deficiency, nitrogen deficiency, and/or C/N imbalance, is genetically characterized by evaluating segregation in first and later generation progeny. The heritability and expression of trait are particularly important if the traits are to be commercially useful.

Thus, in a preferred embodiment, the invention relates to a method for the production of transgenic plants, said method, comprising the steps of:
a) transforming a plant with a nucleic acid encoding a transcription factor as described above; and
b) cultivating the plant cell under condition promoting plant growth and development,
   wherein said transgenic plant has increased tolerance to at least one of sulphur deficiency, nitrogen deficiency and/or C/N imbalance.

In another preferred embodiment, the method of the invention comprises the steps of:
a) transforming a plant with an expression cassette as described above; and
b) cultivating the plant cell under condition promoting plant growth and development.

In a more preferred embodiment, the invention relates to a method for the production of transgenic plants, said method comprises the steps of:
a) transforming a plant with a nucleic acid encoding a transcription factor as described above;
b) selecting a plant cell having increased tolerance to at least one of sulphur deficiency, nitrogen deficiency and/or C/N imbalance; and
c) cultivating the plant cell under condition promoting plant growth and development;
   wherein said transgenic plant has increased tolerance to at least one of sulphur deficiency, nitrogen deficiency and/or C/N imbalance.

In another more preferred embodiment, the method of the invention comprises the steps of:
a) transforming a plant with an expression cassette as described above;
b) selecting a plant cell having increased tolerance to at least one of sulphur deficiency, nitrogen deficiency and/or C/N imbalance and
c) cultivating the plant cell under condition promoting plant growth and development.

The invention also provides a method for selecting a plant that can be used in a breeding process for obtaining a plant with improved yield under optimal or under non optimal conditions for sulphur, nitrogen, and/or C/N balance comprising the step of selecting, in a population of plant, the plants containing the recombinant expression cassette contemplated herein.

In another aspect, the invention relates to a method of plant breeding, e.g., to prepare a crossed fertile transgenic plant.

The method comprises crossing a fertile transgenic plant comprising a particular expression cassette of the invention with itself or with a second plant, e.g., one lacking the particular expression cassette, to prepare the seed of a crossed fertile transgenic plant comprising the particular expression cassette. The seed is then planted to obtain a crossed fertile transgenic plant. The plant may be a monocot or a dicot. In a particular embodiment, the plant is a dicotyledonous plant. The crossed fertile transgenic plant may have the particular expression cassette inherited through a female parent or through a male parent. The second plant may be an inbred plant. The crossed fertile transgenic may be a hybrid.

In another aspect, the invention relates to a method of modulating a plant's yield and/or tolerance to an abiotic stress.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more.

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant.

Stress tolerance in particular is an important factor in determining yield. As explained above, abiotic stress is a primary cause of crop loss worldwide, reducing average yields for most major crop plants by more than 50% (Wang et al. (2003) Planta 218: 1-14). Crop yield may therefore be increased by optimizing tolerance to abiotic stress such as sulphur deficiency, nitrogen deficiency or C/N imbalance. In this regard, the transcription factors of the invention are particularly useful, since overexpression of said transcription factors leads to an increase of plant yield under these conditions.

Thus, in another aspect, the present invention provides a method of modulating a plant's yield and/or tolerance to an abiotic stress comprising modifying the expression of at least one transcription factors as described above in the plant. The yield, and/or tolerance to said abiotic stress of said plant can be increased or decreased as achieved by increasing or decreasing the expression of said transcription factor, respectively. Preferably, the plant's yield and/or tolerance to said abiotic stress is increased by increasing expression of said transcription factor.

In a first embodiment, the present invention relates to a method for increasing or maintaining plant yield under non-optimal conditions for sulphur, nitrogen, and/or C/N balance, said method comprising the step of growing a transformed plant under of non-optimal conditions for sulphur, nitrogen, and/or C/N balance. As used herein, “non-optimal conditions for sulphur, nitrogen, and/or C/N balance” refer to conditions wherein the concentration of sulphur or nitrogen, or the ratio of concentrations of carbon to nitrogen, respectively, is not sufficient for enabling the growth of a normal plant, e.g., an untransformed plant. Said transformed plant is a plant which has been transformed by the expression cassette of the invention. Advantageously, said transformed plant overexpresses one of the transcription factors of the invention. More advantageously, said overexpression leads to an increase in the yield obtained from the transformed plants grown under non-optimal conditions for sulphur, nitrogen, and/or C/N balance, as compared to a control plant not containing the expression cassette of the invention, grown under the same conditions. Alternatively, the yield obtained from the transgenic plants grown under said non-optimal conditions is maintained by said overexpression, as compared to the same transgenic plants grown under optimal conditions for sulphur, nitrogen, and/or C/N balance.

In a preferred embodiment, the invention thus relates to a method for increasing or maintaining plant yield under non-optimal conditions for sulphur, nitrogen, and/or C/N balance, said method comprising a step of growing a transformed plant as contemplated herein, i.e., containing an expression cassette of the invention, under non-optimal conditions for sulphur, nitrogen, and/or C/N balance, wherein:
the yield obtained from said transformed plant grown under said non-optimal conditions is increased as compared to the yield obtained from a plant containing said expression cassette grown under said non-optimal conditions, or
the yield obtained from said transformed plants grown under said non-optimal conditions is maintained as compared to the yield obtained from said transformed plant grown in optimal conditions for sulphur, nitrogen, and/or C/N balance.

Preferably, said method for increasing or maintaining plant yield under non-optimal conditions for sulphur, nitrogen, and/or C/N balance, comprises the prior step of sowing transformed plant seeds.

In a more preferred embodiment, the method for increasing or maintaining plant yield under non-optimal conditions for sulphur, nitrogen, and/or C/N balance, said method comprising a step of growing a transformed plant containing an expression cassette, under non-optimal conditions for sulphur, nitrogen, and/or C/N balance, wherein said expression cassette comprises at least one polynucleotide comprising a nucleotide sequence selected from the group consisting of: (a) a nucleotide sequence that encodes a polypeptide having the amino acid sequence set forth in SEQ ID NOs. 13-24; and (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24, and wherein:
the yield obtained from said transformed plant grown under said non-optimal conditions is increased as compared to the yield obtained from a plant containing said expression cassette grown under said non-optimal conditions, or
the yield obtained from said transformed plants grown under said non-optimal conditions is maintained as compared to the yield obtained from said transformed plant grown in optimal conditions for sulphur, nitrogen, and/or C/N balance.

In an even preferred embodiment, the method for increasing or maintaining plant yield under non-optimal conditions for sulphur, nitrogen, and/or C/N balance, said method comprising a step of growing a transformed plant containing an expression cassette, under non-optimal conditions for sulphur, nitrogen, and/or C/N balance, wherein said expression cassette comprises at least one polynucleotide comprising a nucleotide sequence selected from the group consisting of: (a) a nucleotide sequence that encodes a polypeptide having the amino acid sequence set forth in SEQ ID NOs. 13-24; and (b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24, by conducting a global optimal alignment over the whole length of the respective sequences SEQ ID NOs 13-24, for example by using the algorithm of Needleman and Wunsch (J. Mol. Biol, 48(3): 443-453, 1972), and wherein:
the yield obtained from said transformed plant grown under said non-optimal conditions is increased as compared to the yield obtained from a plant containing said expression cassette grown under said non-optimal conditions, or
the yield obtained from said transformed plants grown under said non-optimal conditions is maintained as compared to the yield obtained from said transformed plant grown in optimal conditions for sulphur, nitrogen, and/or C/N balance.

In another embodiment, the invention also relates to a method for increasing plant yield under optimal conditions for sulphur, nitrogen, and/or C/N balance. Said transformed plant is a plant which has been transformed by the expression cassette of the invention. Advantageously, said transformed plant overexpresses one of the transcription factors of the invention. More advantageously, said overexpression leads to an increase in the yield obtained from the transformed plants grown under optimal conditions for sulphur, nitrogen, and/or C/N balance, as compared to a control plant not containing the expression cassette of the invention, grown under the same conditions.

In a preferred embodiment, the invention thus relates to a method for increasing or maintaining plant yield under optimal conditions for sulphur, nitrogen, and/or C/N balance, said method comprising a step of growing a transformed plant as contemplated herein, i.e., containing an expression cassette of the invention, under conditions of optimal conditions for sulphur, nitrogen, and/or C/N balance, wherein the yield obtained from said grown transformed plant is increased as compared to the yield obtained from a plant not containing said expression cassette and grown under said optimal conditions.

Preferably, said method for increasing or maintaining plant yield under optimal conditions for sulphur, nitrogen, and/or C/N balance, comprises the prior step of sowing transformed plant seeds.

The practice of the invention employs, unless other otherwise indicated, conventional techniques or protein chemistry, molecular virology, microbiology, recombinant DNA technology, and pharmacology, which are within the skill of the art. Such techniques are explained fully in the literature. (See Ausubel et al., Short Protocols in Molecular Biology, Current Protocols; 5th Ed., 2002; Remington's Pharmaceutical Sciences, 17th ed., Mack Publishing Co., Easton, Pa., 1985; and Sambrook et al., Molecular Cloning: A Laboratory Manual, Cold Spring Harbor Laboratory Press; 3rd Ed., 2001). The nomenclatures used in connection with, and the laboratory procedures and techniques of, molecular and cellular biology, protein biochemistry, enzymology and medicinal and pharmaceutical chemistry described herein are those well-known and commonly used in the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The following examples illustrate certain aspects of the invention. The examples in no way limit the invention.

### EXAMPLES

### Example 1: Screen of Arabidopsis Transcription Factors Genes

Nutrient Deficiency Screenings

To identify transcription factors that promote an enhancement of plant's growth and tolerance under different N, C/N and S nutrient limitations, three screenings were carried out under different conditions: nitrogen deficiency, carbon/nitrogen imbalance and sulphur deficiency. The Arabidopsis TRANSPLANTA collection (Coego et al., 2014) consisting of transgenic lines with transcription factor-inducible expression was used in the screenings where ecotype Columbia (Col-0) was the wild-type control. Each one of the transgenic Arabidopsis lines in the collection expresses a single Arabidopsis transcription factor under the control of a ß-estradiol inducible promoter. The cDNA of each transcription factor was previously cloned into the pER8GW binary vector and verified by full-length sequencing. These constructs were used for genetic transformation of Arabidopsis thaliana plants, which were further selected until T3 homozygous lines (Coego et al., 2014).

Low Nitrogen Screening

In order to identify transcription factors that enable plant growth under low nitrogen conditions, seeds from control plants (Col-0) and at least two independent T3 homozygous transgenic lines of each transcription factor (8-10 seeds per line, TRANSPLANTA collection) were sterilized (using ethanol 70% for 2 min, a solution containing sodium hypochlorite 0.25%, SDS 1% and MilliQ water for 12 min, followed by five washes with sterilized MilliQ water), vernalized for 2 days at 4° C. and then plated onto two sets of Petri dishes containing control (30 mM N, 1% sucrose) and low nitrogen medium (0.1 mM N, 1% sucrose), both supplemented with 10 μM ß-estradiol. After 5 days of growth in a culture chamber at 22° C. under long-day conditions (16 h light/8 h darkness) pictures were taken. Tolerant lines were scored as those with more than five green seedlings (out of eight).

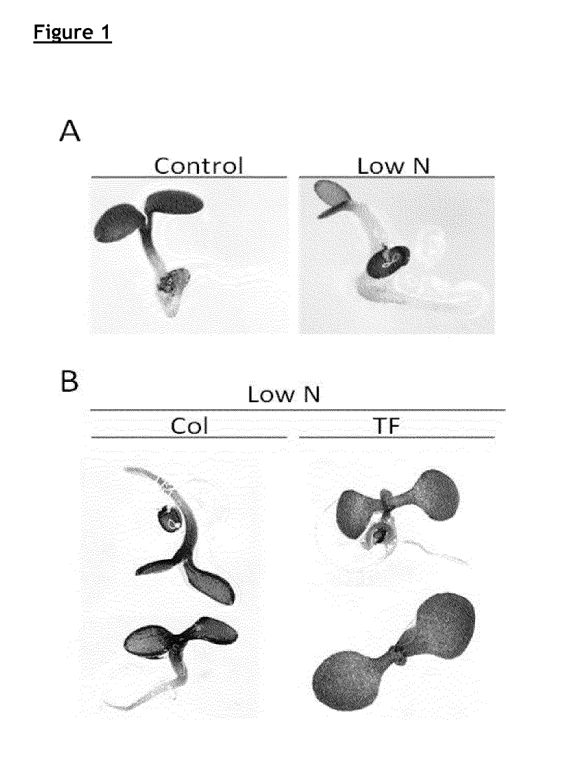

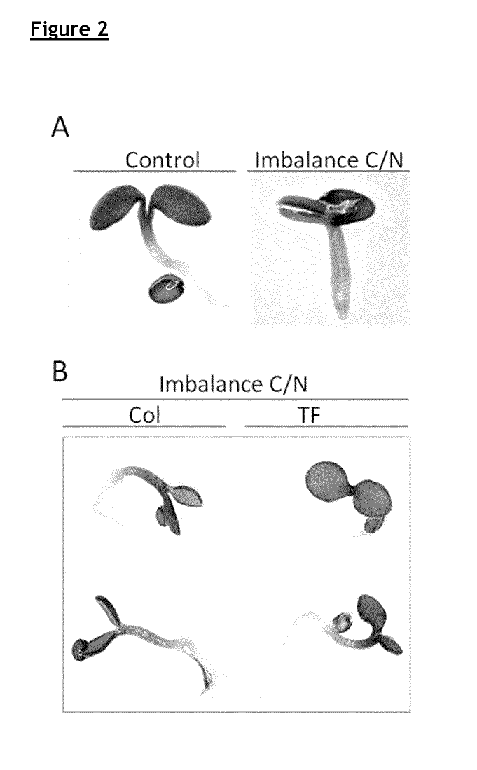

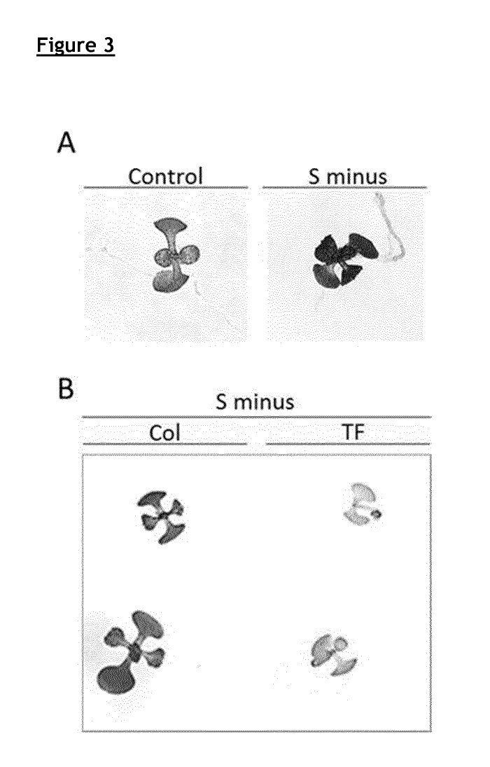

Control plants showed chlorotic leaves and purple leaf edges after 5 days of growth. However, we could identify different transgenic lines corresponding to 11 different transcription factors that were able to grow and displayed a WT phenotype under low N conditions (FIG. 1, Table 1).

Carbon/Nitrogen (C/N) Imbalance Screening

To identify transcription factors enabling plant growth under C/N imbalance conditions (high levels or carbon and low levels nitrogen) a similar screening was conducted. Control plants (Col-0) and T3 homozygous transgenic lines of each transcription factor (8-10 seeds per line of the TRANSPLANTA collection) were grown in Petri dishes using standard (30 mM N, 1% sucrose) and C/N imbalance medium (0.1 mM N, 300 mM Glucose), both supplemented with 10 μM ß-estradiol. After 7 days of growth in a culture chamber at 22° C. under long-day conditions (16 h light/8 h darkness) lines with more than five green seedlings were scored as tolerant (FIG. 2).

As shown in FIG. 2, after 7 days of growth, wild-type control plants exhibit a strong growth inhibition with leaves with increased purple colour. Nevertheless, among the transcription factor-overexpressing plants, several lines corresponding to different transcription factors were able to grow and rescue a WT phenotype under imbalanced C/N conditions (FIG. 2, Table 1).

Sulphur Starvation Screening

In this case, the goal was to identify transcription factors that allowed plant growth under Sulphur starvation conditions, thus a similar genetic strategy was conducted. We used as plant material control plants (Col-0) and T3 homozygous transgenic lines from the TRANSPLANTA collection (8-10 seeds per line). Seeds were sterilized and germinated as previously described and grown in Petri dishes under control conditions (2.5 mM KH₂PO₄, 2 mM MgSO₄, 1% sucrose) and S-minus medium (2.5 mM KH₂PO₄, 2 mM MgCl₂, 1% sucrose) both supplemented with 10 μM ß-estradiol. After 9 days of growth in a culture chamber at 22° C. under long-day conditions (16 h light/8 h darkness) plants were photographed and tolerant lines were scored as those where more than five green seedlings could grow (FIG. 3).

After 9 days of growth in S-minus medium, control plants exhibit purple-dark leaves that were even more evident 2-5 days later (FIG. 3). In those conditions several transgenic lines corresponding to 4 different transcription factors could grow and rescue the WT phenotype (FIG. 3, Table 1).

### Example 2—Cloning of Transcription Factors Downstream the Rice Actin Promoter and Transformation into Maize

Each of the 12 Arabidopsis coding sequences identified in the above screens (see table 1) was codon optimized for expression in maize and cloned into the pUC57 vector. The optimized sequences were then cloned between a rice Actin promoter+intron (McElroy et al 1990), and an Arabidopsis Sac66 polyadenylation sequence (Jenkins et al (1999)), into the destination binary plasmid pBIOS3092. The binary vector pBIOS3092 is a derivative of pSB12 (Komari et al. (1996)) containing a rice actin promoter+actin intron-selectable marker-nos terminator chimeric gene for selection of maize transformants and a Zoanthus Green reporter gene driven by the wheat High Molecular Weight Glutenin promoter.

The binary plasmids containing the cloned expression cassettes were transferred into agrobacteria LBA4404 (pSB1) according to Komari et al (1996). Maize cultivar A188 was transformed with these agrobacterial strains essentially as described by Ishida et al (1996).

Analysis of the transformed maize plants indicated that some plants overexpressed the transcription factors.

### Example 3—Maize Field Trials for NUE Tolerance

A—Field Trials

Hybrids with a tester line were obtained from T3 plants issued from the transgenic maize line made according to example 2.

The transformants (T0) plant was first crossed with the A188 line thereby producing T1 plants. T1 plants were then self-pollinated twice, producing T3 plants which are homozygous lines containing the transgene. These T3 plants were then crossed with the tester line thereby leading to a hybrid. This hybrid is at a T4 level with regards to the transformation step and is heterozygous for the transgene. These hybrid plants are used in field experiments.

Control hybrids are obtained as follows:

Control Equiv corresponds to a cross between a A188 line (the line used for transformation) and the tester line.

Control Null segregants correspond to a cross between a null segregant (isolated after the second self-pollination of the T1 plants) and the tester line. Said null segregant is a homozygous line which does not bear the transgene. Although the null segregant theoretically presents the same genome as A188, it has undergone in vitro culture (via the steps of callus differentiation and regeneration) and may thus present mutations (either genetic or epigenetic) with regards to a A188 line that has not undergone in vitro culture.

These two control lines are used to avoid any effect that could be due to mutations (genetic or epigenetic) coming from in vitro culture steps.

Yield is calculated as follows:

During harvest, grain weight and grain moisture are measured using on-board equipment on the combine harvester.

Grain weight is then normalized to moisture at 15%, using the following formula:

Normalized grain weight=measured grain weight×(100−measured moisture (as a percentage))/85 (which is 100−normalized moisture at 15%).

As an example, if the measured grain moisture is 25%, the normalized grain weight will be: normalized grain weight=measured grain weight×75/85.

Yield is then expressed in a conventional unit (such as quintal per hectare).

B—Experimental Design:

Field trials were conducted on different locations.

Plants were sown between mid-May and mid-June. Harvest was between mid-September and the mid-October at the latest.

The experimental block comprises 5 to 6 replicates. The experimental design is a Randomized complete block or Lattice in both optimal locations and non-optimal (N-stress) locations. Each replicate comprised two row plots with about up to 60 plants per plot at a density of 70 000 plants/ha.

Controls were used in this experiment were those described above (null segregant and a control equivalent (A188 crossed with the tester line).

An optimal location for nitrogen treatment is a location where 100% of the recommended nitrogen rate is applied

A non-optimal location for nitrogen treatment is a location where only 30% of the total recommended nitrogen rate is applied.

The nitrogen stress intensity is evaluated by measuring the yield lost between the nitrogen stress treatment (30% of recommended nitrogen rate applied) and a reference treatment fertilized with 100% of recommended nitrogen rate.

A yield loss of −30% is targeted with a common distribution of the N-stress location between −10% and −40% of yield.

Overexpression of the transcription factors alleviates said yield loss, partially or totally.

TABLE 3

| | | | | % similarity | |
|---|---|---|---|---|---|
| | | | | between | |
| | | | | the | |
| | | | | Arabidopsis | |
| | | Orthologous | | and corn | Orthologous |
| Arabidopsis | | Corn | SEQ | proteins | Rice |
| protein | Gene | peptide | ID | (according | peptide |
| sequence | family | sequence | NO. | to BBMH) | sequence |
| AT5G45580 | G2-like | GRMZM2G125704_P02 | 42 | 56.49 | Os02g47190.1 |
| AT1G67260 | TCP | AC233950.1_FGP002 | 43 | 55.68 | Os03g49880.1 |
| AT1G66230 | MYB | GRMZM2G048910_P01 | 44 | 71.43 | Os09g23620.1 |
| AT3G50650 | GRAS | GRMZM2G104342_P01 | 45 | 57.89 | Os03g51330.1 |
| AT3G23240 | AP2- | AC233933.1_FGP001 | 46 | 54.74 | Os07g22770.1 |
| | EREBP | | | | |
| AT2G33880 | HB | GRMZM2G409881_P01 | 47 | 64.29 | Os05g48990.1 |
| AT4G17490 | AP2- | GRMZM5G805505_P01 | 48 | 54.89 | Os04g46240.1 |
| | EREBP | | | | |
| AT5G43290 | WRKY | AC165171.2_FGP002 | 49 | 65.81 | Os01g74140.1 |
| AT2G45660 | MADS | GRMZM2G070034_P01 | 50 | 72.02 | Os10g39130.1 |
| AT4G05100 | MYB | GRMZM2G031323_P01 | 51 | 82.01 | Os09g36730.1 |
| AT5G64810 | WRKY | GRMZM2G101405_P01 | 52 | 62.59 | Os05g46020.1 |
| AT5G13790 | MADS | GRMZM2G160565_P01 | 53 | 56.20 | Os02g45770.1 |

| | | % | | | % |
|---|---|---|---|---|---|
| | | similarity | | | similarity |
| | | between | | | between |
| | | the | | | the |
| | | Arabidopsis | | | Arabidopsis |
| | | and | | | and |
| | | rice | | | Brachypodium |
| | | proteins | Orthologous | | proteins |
| Arabidopsis | SEQ | (according | Brachypodium | SEQ | (according |
| protein | ID | to | peptide | ID | to |
| sequence | NO. | BBMH) | sequence | NO. | BBMH) |
| AT5G45580 | 54 | 62.20 | Bradi5g20520.1 | 66 | 59.84 |
| AT1G67260 | 55 | 58.55 | Bradi1g11060.1 | 67 | 55.48 |
| AT1G66230 | 56 | 70.25 | Bradi4g29800.1 | 68 | 66.42 |
| AT3G50650 | 57 | 58.09 | Bradi1g10330.1 | 69 | 60.21 |
| AT3G23240 | 58 | 54.78 | Bradi1g00670.1 | 70 | 71.24 |
| AT2G33880 | 59 | 59.17 | Bradi1g63680.1 | 71 | 43.85 |
| AT4G17490 | 60 | 50.66 | Bradi5g17490.1 | 72 | 57.73 |
| AT5G43290 | 61 | 50.37 | Bradi1g59180.1 | 73 | 77.42 |
| AT2G45660 | 62 | 69.91 | Bradi3g32090.1 | 74 | 72.52 |
| AT4G05100 | 63 | 65.02 | Bradi3g46910.1 | 75 | 89.39 |
| AT5G64810 | 64 | 69.67 | Bradi2g18530.1 | 76 | 74.07 |
| AT5G13790 | 65 | 57.81 | Bradi3g51800.2 | 77 | 66.47 |

### Example 4: Test of Zea mays Orthologous Sequences in Low Nitrogen Screening

The maize transcription factors ZmAGL20 (SEQ ID NO: 50), ZmG2like (SEQ ID NO: 42), ZmERF1 (SEQ ID NO: 46), ZmMYB20 (SEQ ID NO: 44), ZmAGL15 (SEQ ID NO: 53) and ZmWRKY51 (SEQ ID NO: 52) were screened out under nitrogen deficiency condition.

Each one of the maize transcription factor is expressed under the control of a ß-estradiol inducible promoter as described in example 1. The cDNA of each transcription factor was previously cloned into the pER8GW binary vector and verified by full-length sequencing. These constructs were used for genetic transformation of Arabidopsis thaliana plants.

Low Nitrogen Screening:

In order to identify transcription factors that enable plant growth under low nitrogen, 50 to 55 seeds of control plants (GFP expressed in the pER8 ß-estradiol inducible system) and from transgenic lines expressing each a selected transcription factor in the same system were analyzed as described in the example 1. In a first assay, seeds were grown in N-supplied standard medium under inducible conditions (supplemented with 10 μM ß-estradiol) to check if expression of the corresponding factors had any deleterious effect on seed germination and/or seedling development. The results presented in Table 4 show that all the TF-expressing lines have similar behavior as the control GFP-expressing line, both in percentage of germination and in displaying normal seed development.

In a second assay these lines were plated on low-nitrogen medium under inducible conditions. As observed in standard medium, seed germination rates were not affected (Table 4). However, seedling development was severely compromised in the control line (expressing GFP) after five days of growth under nitrogen limited conditions. This line shows both impaired cotyledon development and reduced production of photosynthetic pigments, in parallel with increased anthocyanin accumulation. By contrast, all the TF-expressing lines displayed enhanced seedling development and better performance (up to nearly three times, see Table 4) than the control line under nitrogen-limited conditions.

These results show that Arabidopsis plants expressing selected transcription factors perform better under low-N conditions than a control plant expressing the GFP.

### REFERENCES

Chen W, Provart N J, Glazebrook J, Katagiri F, Chang H S, Eulgem T, Mauch F, Luan S, Zou G, Whitham S A, Budworth P R, Tao Y, Xie Z, Chen X, Lam S, Kreps J A, Harper J F, Si-Ammour A, Mauch-Mani B, Heinlein M, Kobayashi K, Hohn T, Dangl J L, Wang X, Zhu T. (2002) Expression profile matrix of Arabidopsis transcription factor genes suggests their putative functions in response to environmental stresses. Plant Cell. March; 14(3):559-74.
Chinnusamy V, Schumaker K, Zhu J K (2004). Molecular genetic perspectives on cross-talk and specificity in abiotic stress signalling in plants. J Exp Bot. January; 55 (395):225-36.
Coego, A., Brizuela, E., Castillejo, P., Ruíz, S., Koncz, C., del Pozo, J C., Piñeiro, M., Jarillo, J A., Paz-Ares, J., León, J and The TRANSPLANTA Consortium (2014). The TRANSPLANTA collection of Arabidopsis lines: a resource for functional analysis of transcription factors based on their conditional overexpression. The Plant Journal. 77: 944-953.
Coruzzi, G. M. and Zhou, L. (2001) Carbon and nitrogen sensing and signaling in plants: emerging ‘matrix effects’. Curr. Opin. Plant Biol. 4:247-253.
Dolferus R, Jacobs M, Peacock W J, Dennis E S. (1994) Differential interactions of promoter elements in stress responses of the Arabidopsis Adh gene. Plant Physiol. 1994 August; 105(4):1075-87.
Fernández-Muñoz, R. Y Cuartero, J. (2005) Situación actual de la Mejora Vegetal en tomate para fresco. En: El cultivo de tomate para fresco. Ministerio de Agricultura, Pesca y Alimentación. Madrid. Páginas: 59-76.
Fernie A, Tadmor Y and Zamir D. (2006) Natural genetic variation for improving crop quality. Current opinion in Plant Biology 9:196-202.
Foolad, M. R. (2007). Genome Mapping and Molecular Breeding of Tomato. International Journal of Plant Genomics, 2007.
Gao G., Zhong Y., Guo A., Zhu Q., Tang W., Zheng W., Gu X., Wei. L and Luo J. (2006) DRTF: a database of rice transcription factors. Bioinformatics 22: 1286-1287.
Gibon, Y., Biasing, O. E., Palacios-Rojas, N., Pankovic, D., Hendriks, J. H. M., Fisahn, J. et al. (2004) Adjustment of diurnal starch turnover to short days: depletion of sugar during the night leads to a temporary inhibition of carbohydrate utilization, accumulation of sugars and post-translational activation of ADP-glucose pyrophosphorylase in the following light period. Plant J. 39: 847-862
Ishiguro, S. and Nakamura K. (1994) Characterization of a cDNA encoding a novel DNA-binding protein, SPF1, that recognizes SP8 sequences in the 5′ upstream regions of genes coding for sporamin and beta-amylase from sweet potato. Mol Gen Genet. 244:563-571.
Jin J P, Zhang H, Kong L, Gao G and Luo J C. (2014). PlantTFDB 3.0: a portal for the functional and evolutionary study of plant transcription factors. Nucleic Acids Research, 42(D1):D1182-D1187.
Kiba, T., Kudo, T., Kojima, M. and Sakakibara, H. (2011) Hormonal control of nitrogen acquisition: roles of auxin, abscisic acid, and cytokinin. J. Exp. Bot. 62: 1399-1409
Kim S. Y. Chung H. J. and Thomas T. L. (2002) Isolation of a novel class of bZIP transcription factors that interact with ABA-responsive and embryo-specification elements in the Dc3 promoter using a modified yeast one-hybrid system. The Plant J. 11:1237-1251.
Klotke, J., Kopka, J., Gatzke, N. and Heyer, A. G. (2004) Impact of soluble sugar concentrations on the acquisition of freezing tolerance in accessions of Arabidopsis thaliana with contrasting cold adaptation—evidence for a role of raffinose in cold acclimation. Plant Cell Environ. 27: 1395-1404.
Martin, C. and Paz-Ares, J. (1997) MYB transcription factors in plants. Trends Genet 13: 67-73.
Martin, T., Oswald, O. and Graham, I. A. (2002) Arabidopsis seedling growth, storage lipid mobilization, and photosynthetic gene expression are regulated by carbon:nitrogen availability. Plant Physiol. 128:472-481.
Matsuoka M., Tada Y, Fujimura T, Kano-Murakami Y. (1993), Tissue-specific light-regulated expression directed by the promoter of a C4 gene, maize pyruvate, orthophosphate dikinase, in a C3 plant, rice. Proc Natl Acad Sci USA., October 15; 90 (20):9586-90.
Miller, A. J., Fan, X. R., Orsel, M., Smith, S. J. and Wells, D. M. (2007) Nitrate transport and signalling. J. Exp. Bot. 58: 2297-2306.
Naika M., Shameer K., Mathew O. K., Gowda R. and Sowdhamini R. (2013) STIFDB2: An Updated Version of Plant Stress-Responsive Transcription Factor DataBase with Additional Stress Signals, Stress-Responsive Transcription Factor Binding Sites and Stress-Responsive Genes in Arabidopsis and Rice. Plant Cell Physiol. 54(2):e8(1-15).
Qu L. J. and Zhu Y. X. (2006) Transcription factors families in Arabidopsis: major progress and outstanding issues for future research. Current Opinion in Plant Biology 9: 544-549.
Paterson A. H., Lander E. S., Hewitt J. D., S. Peterson, Lincoln S. E, and Tanksley S. D., “Resolution of quantitative traits into Mendelian factors by using a complete linkage map of restriction fragment length polymorphisms,” Nature, vol. 335, no. 6192, pp. 721-726, 1988.
Perez-Rodriguez P., Riano-Pachon D. M. Guedes Correa L. G., Rensing S. A, Kersten B., Mueller-Roeber B. PlnTFDB: updated content and new features of the plant transcription factor database. Nucleic Acids Research 2009.
Ribaut J M, de Vicente M C, Delannay X (2010) Molecular breeding in developing countries: challenges and perspectives. Current Opinion in Plant Biology 13:1-6.
Riechmann J. L. and Meyerowitz E. M. (1997) MADS domain proteins in plant development. Biol Chem 378: 1079-1101.
Riechmann J. L. and Meyerowitz E. M. (1998) The AP2/EREBP family of plant transcription factors. Biol Chem 379: 633-646.
Riechmann J. L., Heard J., Martin G., Reuber L., Jiang C., Keddie J., Adam L., Pineda O., Ratcliffe O. J., Samaha R. R., Creelman R., Pilgrim M., Broun P., Zhang J. Z., Ghandehari D., Sherman B. K., Yu G. (2000) Arabidopsis transcription factors: genome-wide comparative analysis among eukaryotes. Science. December 15; 290 (5499):2105-10.
Roitsch, T. and Gonzalez, M. C. (2004) Function and regulation of plant invertases: sweet sensations. Trends Plant Sci. 9: 606-613.
Smith, A. M. and Stitt, M. (2007) Coordination of carbon supply and plant growth. Plant Cell Environ. 30: 1126-1149
Sato, T., Maekawa, S., Yasuda, S. and Yamaguchi, J. (2011b) Carbon and nitrogen metabolism regulated by the ubiquitin-proteasome system. Plant Signal. Behav. 6: 1465-1468.
Sulpice, R., Nikoloski, Z., Tschoep, H., Antonio, C., Kleessen, S., Larhlimi, A. et al. (2013) Impact of the carbon and nitrogen supply on relationships and connectivity between metabolism and biomass in a broad panel of Arabidopsis accessions. Plant Physiol. 162: 347-363.
Sambrook J, Fritsch E F, Maniatis (1989) in cloning: a laboratory Manual (Cold Spring Harbor Lab Press. Cold Spring Harbor, N.Y.) 2nd Ed.
Tuberosa R and Salvi S (2006). Genomics-based approaches to improve drought tolerance of crops. Trends in Plant Science. 11:1360-1385.
Vij S and Tyagi A (2007) Emerging trends in the functional genomics of the abiotic stress response in crop plants. Plant Biotechnology Journal. 5: 361-380.
Wang W, Vinocour B and Altman A (2003) Plant responses to drought, salinity and extreme temperatures: towards genetic engineering for stress tolerance. Planta 218; 1-14.
Wang X, Jiang G L, Green M, Scott R A, Hyten D L, Cregan P B (2012) Quantitative trait locus analysis of saturated fatty acids in a population of recombinant inbred lines of soybean. Mol. Breeding 30:1163-1179.
Yamaguchi-Shinozaki K and Shinozaki K (2006). Transcriptional Regulatory Networks in Cellular Responses and Tolerance to Dehydration and Cold Stresses. Annu Rev Plant Biol. March 3; 781-795.
Yu W., Qing Z., Lei G., Xiao-Min Y., Ping F., David J., and Cheng-Bin X (2010). Isolation and characterization of low-sulphur-tolerant mutants of Arabidopsis. Journal of Experimental Botany. 61: 34
Zhang H., Scheirer D. C., Fowle W. H. and Goodman H. M. (1992) Expression of antisense or sense RNA of an ankyrin repeat-containing gene blocks chloroplast differentiation in arabidopsis. Plant Cell 4; 1575-1588.
Zuber H, Davidian J C, Wirtz M, Hell R, Belghazi M, Thompson R, Gallardo K (2010). Sultr4; 1 mutant seeds of Arabidopsis have an enhanced sulphate content and modified proteome suggesting metabolic adaptations to altered sulphate compartmentalization. BMC Plant Biol. 28; 10:78.07-3422.

1. An expression cassette comprising at least one polynucleotide comprising a nucleotide sequence selected from the group consisting of:
a) a nucleotide sequence that encodes a polypeptide having the amino acid sequence set forth in SEQ ID NOs. 13-24; and
b) a nucleic acid sequence that encodes a polypeptide having the amino acid sequence sharing at least 50%, preferably at least 55%, 60%, 65%, 70%, 75%, 80%, 85%, especially preferably at least 90%, 95%, 97%, 98%, 99%, or more of sequence identity with a sequence selected in the group consisting of SEQ ID NOs. 13-24.

2. An expression cassette according to claim 1, wherein said expression cassette has a sequence selected from the sequences represented by SEQ ID NOs. 30-41.

3. A plant cell comprising the expression cassette according to claim 1.

4. A transgenic plant comprising a plant cell according to claim 3.

5. The transgenic plant of claim 4, wherein said plant is a monocot or a dicot.

6. The transgenic plant of claim 4, wherein said plant is selected from the group consisting of: maize, soybean, sunflower, sorghum, canola, wheat, alfalfa, cotton, rice, barley, millet and sugar cane.

7. A transgenic seed from the transgenic plant of claim 4.

8. A method for the production of a transgenic plant, said method comprising the transformation of a plant by an expression cassette according to claim 1.

9. A method for increasing or maintaining plant yield under non-optimal conditions for sulphur, nitrogen, and/or C/N balance, said method comprising a step of growing a transgenic plant according to claim 4 under conditions of non-optimal conditions for sulphur, nitrogen, and/or C/N balance, wherein:
the yield obtained from said transgenic plant grown under said non-optimal conditions is increased as compared to the yield obtained from a plant not containing the expression cassette, grown under said non-optimal conditions, or
the yield obtained from said transgenic plants grown under said non-optimal conditions is maintained as compared to the yield obtained from a transgenic plant according to claim 4, and grown in optimal conditions for sulphur, nitrogen, and/or C/N balance.

10. A method for increasing or maintaining plant yield under optimal conditions for sulphur, nitrogen, and/or C/N balance, said method comprising a step of growing a transgenic plant according to claim 4 under conditions of optimal conditions for sulphur, nitrogen, and/or C/N balance, wherein the yield obtained from said transgenic plant is increased as compared to the yield obtained from a plant not containing the expression cassette, grown under said optimal conditions.

11. A method for selecting a plant that can be used in a breeding process for obtaining a plant with improved yield under optimal or under non optimal conditions for sulphur, nitrogen, and/or C/N balance comprising the step of selecting, in a population of plant, the plants containing the recombinant expression cassette according to claim 1.

12. A method for identifying a plant with improved yield under optimal or under non optimal conditions for sulphur, nitrogen, and/or C/N balance comprising the step of identifying, in a population of plant, the plants containing the recombinant expression cassette according to claim 1.


SEQUENCE LISTING (89 sequences)

SEQ ID NO: 1 - DNA, 795 bp, Arabidopsis thaliana
  misc_feature: AT5G4558
atgatgactagagatcccaaacctaggctcagatggacagccgatcttcatgatcgcttcgtcgacgccgtcgctaagctcggtggcgctgacaaagcaactcctaaatcggttctgaagctgatgggattaaaaggtttaacactgtaccatctcaagagccatttacagaagtatagacttggtcaacaacaaggcaaaaaacaaaatagaacagaacaaaacaaagaaaatgctggaagttcatatgtgcatttcgacaattgttctcaaggaggaatctccaatgattcaagatttgataaccatcaaagacaaagcgggaatgtaccttttgctgaggcaatgagacatcaggtcgatgcgcaacaacggtttcaagaacaactcgaggtgcagaaaaaactgcagatgagaatggaggcacaaggaaaatatttgttgacattactagagaaagcacagaagagcttaccttgtggtaatgcaggggaaacagacaagggtcaattctcggatttcaatctcgcactttcgggacttgtaggaagtgatcgtaagaacgagaaggcgggtttggttactgatatctcacaccttaatggtggtgattcctctcaagagtttcggttatgtggagaacaagagaaaatagaaaccggagatgcatgtgttaaaccagaatccgggtttgtgcattttgatttgaactccaaaagtgggtatgatctcttgaattgtgggaaatatgggattgaagtgaagccaaatgtgattggagatagacttcaatag

SEQ ID NO: 2 - DNA, 1307 bp, Arabidopsis thaliana
  misc_feature: AT1G67260
atgtcgtcttccaccaatgactacaacgatggtaataacaatggagtgtaccctctctctctttacctttcttcactctctggccatcaagacatcattcataatccctacaaccatcagttaaaagcatctccgggccatatggtatcagcagttcctgaatctctgatcgattacatggcgtttaagtcaaataatgttgtgaatcaacaaggctttgagtttcctgaggtgtcaaaggaaatcaagaaggtggtgaagaaggaccgacatagcaagattcaaacggcacaagggattagagacaggagggttaggctttctattgggattgctcgccaattctttgatcttcaggatatgttggggtttgataaagctagtaaaacgttagactggctgctcaagaagtcaagaaaagccatcaaagaggtcgtacaagcaaaaaacctcaacaatgatgatgaagattttggaaacattggaggcgatgtagaacaagaagaggagaaggaggaggatgacaatggcgataagagcttcgtgtatggtttgagccccgggtacggtgaagaagaagtggtatgtgaggccacgaaggcagggataagaaagaagaagagtgagttgagaaacatctcatcaaaggggctaggagccaaagctagaggaaaagcaaaggagcgaacaaaagagatgatggcctatgataatccagagactgcctctgatattacacaatctgaaatcatggacccattcaagaggtctatagtcttcaatgaaggagaagatatgacacaccttttctacaaggaaccaatcgaggagtttgataatcaagaatctatcttaaccaatatgactctaccaacgaagatgggtcaaagttacaatcaaaataatgggatacttatgttggtagatcagagttctagcagcaactataatacatttctgcctcaaaatttggattatagttatgatcaaaacccttttcatgaccaaaccttatatgtagtcaccgacaaaaatttccccaaaggtttcctataaatctcgacagttttgaaggactatgcatgatcaagtttaaacatgtgacactgagaattagtgtgtggaatgtggaggcgtgcctgccgtggtgtctatttgattcatagtttgtgatttggccatccaggtatgaggagaatatatcctactttcatattattgtctatatagaagatatatacacatttatatattggtgtatttattctcttactcggaggatgttttgcaatgatgctttttgaagc

SEQ ID NO: 3 - DNA, 1186 bp, Arabidopsis thaliana
  misc_feature: AT1G66230
catacatcagtatgcatatatataggtggttgcaaggcttacccaaatctctcttcaacttgccataacatttactctctttggacaaagacacagacagagagatagataaaaacagagaattgtcatagaaccaattcagagagaacgagagagagagagagagaaatggggagacaaccatgctgtgacaaagtagggttgaagaaaggaccatggactgcagaagaggataggaagctcataaacttcatccttaccaatggacaatgttgttggagagctgttcctaagctttctggtcttcttaggtgtggcaagagttgcagacttcgttggactaactatcttagaccagaccttaagagaggtcttctctctgattacgaagagaagatggtcattgatctccattcccagcttggaaacaggtggtcaaagatagcttctcatttaccaggaagaacagacaacgaaatcaagaatcattggaacactcacatcaagaagaagttgaggaaaatggggattgatcctcttacacataaaccactctctatcgtcgaaaaagaagacgaagaacccttaaagaagctacagaataatacagttccttttcaagaaacaatggagcgtcctttagagaacaacatcaagaacatatcaagacttgaagagtctttaggtgatgatcaattcatggagataaatcttgagtatggtgtcgaagatgtccctcttattgaaacagagtctttagaccttatctgcagcaattcaacaatgtcttcatccacgtccacatcttcgcattcttctaatgattcgagtttcttgaaggatttgcagttcccggagttcgagtggtccgactatggtaatagtaataatgataataataatggtgtggacaacattatagagaacaatatgatgagcctgtgggaaattagtgactttagcagtttggatttgctgcttaatgatgagagttcttccacttttgggttgttttgaattcattcttaagaatatggtttcttttgtaaaagaatggaaactatggggtgggggaaacagaaaaacctaaatacccttcgttactctagtttttgagggatttgagagaacgtttttttacttgtgagaaatttagaaacaaaaataaagtttatttattttgtac

SEQ ID NO: 4 - DNA, 1953 bp, Arabidopsis thaliana
  misc_feature: AT3G50650
aaaatctctccggtttcattcatatccctctctctctctgggccttactcaatcaccaccaaataacagacaaaaaaacgccttcatcgccggcgaaagtgagtcggagaatggcgtatatgtgcaccgacagcggaaacttaatggctatagcacaacaactcatcaaacagaagcagcaacaacaatcacaacatcaacaacaagaagaacaagaacaagaacccaacccctggcccaatccatccttcgggtttactcttcccggttcgggtttctccgacccgtttcaagtaaccaacgacccaggttttcactttccacacctggagcatcatcaaaacgcagccgtcgcttccgaggagtttgactccgacgagtggatggagagtttgatcaacggtggagatgcgtcacaaaccaatccagacttcccaatctacggccatgatccattcgtctcattcccgagtcgactcagtgctccttcatatctcaaccgagtcaacaaagatgactcagcgagtcaacaactccctccgccaccagcttccaccgctatatggtctccatctccgccatcaccacaacatcctcctcctcccccgccgcagccggattttgatctaaaccagccgatttttaaagctatccacgattatgcacgtaaaccggaaactaaaccggacacactaattcggatcaaagaatccgtgtccgaatcaggcgacccgatacaacgagtcgggtattacttcgcagaagctctgtctcataaagaaacagagtctccgtcgtcatcgtcgtcgtcatccctagaagatttcattctctcatacaaaaccttaaacgacgcatgtccatattcaaagttcgctcatttaacagcgaatcaagcaattctcgaagctacgaatcaatcaaacaacattcacatagtcgatttcgggatttttcaaggtattcaatggtctgctctgttacaagctttagcgacccgttcttctggtaaacccacccggatccggatttcgggtatacccgctccgtctctaggagactcaccgggaccgtctctgatcgctacgggaaaccgtctccgtgatttcgcggcgattttggatctcaatttcgagttttatccggttctgactccgattcaattactaaacgggtcaagttttcgggttgacccggatgaggttttggttgtgaattttatgcttgagctttataagcttctagatgaaacagcgacgactgttggtacggcgctccggttagctagatcgttaaacccgaggattgtgacgcttggagaatacgaagtcagtttaaaccgggttgaatttgctaaccgggttaagaactctctccggttttattccgcggtttttgaatcgcttgaaccgaatttggatcgagattcgaaagaaaggctgagagtggagagagtgttgttcggtaggaggattatggatttggtccgatcagatgatgataataataaaccgggaacccggtttgggttaatggaggagaaagaacaatggagagtgttgatggagaaagctggatttgagccggttaaaccgagtaattacgcggttagccaagcgaagctgctactatggaactacaattatagtacattgtattcacttgttgaatcggagccaggtttcatctccttggcttggaacaatgtgcctctcctcaccgtttcctcttggcgttgactacttggtccgataagttaatctagtattttgagttagcttttagaattgaattgtttggggttagatttggatgtttaattagtctctagcctattctcttactcttttttatctagtgcttggagtgatgatggtttgtcgtttatgttcatttgtaatatatattgtatgtaacatttgactagatttctttggtataaacgaaagaagc

SEQ ID NO: 5 - DNA, 862 bp, Arabidopsis thaliana
  misc_feature: AT3G23240
aaaccgaaaacagagtagtcaagaaacagagtattttttctacatggatccacttttaattcagtccccattctccggcttctcaccggaatattctatcggatcttctccagaatctttctcatcctcttcttctaacaattactctcttcccttcaacgagaacgactcagaggaaatgtttctctacggtctaatcgagcaatccacgcaacaaacctatatcgactcggaacttcaagaccttccgatcaaatccgtaagctcaagaaagtcagagaagtcttacagaggcgtaagacgacggccatgggggaaattcgcggcggagataagagattcgactagaaacggtattagggtttggctcgggacgttcgaaagcgcggaagaggcggctttagcatacgatcaagccgctttctcgatgagagggtcctcggcgattctcaacttttcggcggagagagttcaagagtcgctttcggagattaagtgtacctacgaggatggttgttctccggttgtggcgttgaagaggaaacactcgatgagacggagaatgaccaataagaaaacgaaagatagtgactttgatcaccgctccgtgaagttagataatgtagttgtctttgaggatttgggagaacagtaccttgaggagcttttgggttcttctgaaaatagtgggacttggtgaaagattaggatttgtgttagggaccttaagtttgaagtggttgattaattttaaccctaatatgttttttgtttgcttaaatatttgattctattgagaaacatcgaaaacagtttgtatgtatttttgtgatacttgactttatcaatttcaagttagtgc

SEQ ID NO: 6 - DNA, 1527 bp, Arabidopsis thaliana
  misc_feature: AT2G33880
gaggtcgtaacccttttgccctattcacatttttatttatctttccatttagccattctgttccctgtctcttcctcctctctttttgacacatcacatcatcatcacatcatcattcaacatcaatcatcatcatatgcatacacatacatctgtgttctgcggatcgagttaattagttatggcttcttcgaatagacactggccaagcatgttcaagtccaaacctcatccccatcaatggcaacatgacatcaactctcctctcttgccttctgcttctcaccgatcttctcctttctcttcaggatgtgaagtggagaggagtccagagccaaaaccaagatggaatccaaagccagagcagattcggatacttgaagcaatctttaactccgggatggtgaatcctccaagagaggagatcaggaggattagggctcagcttcaagaatacggccaagtcggtgatgctaacgtcttctactggttccaaaaccgtaagtcccgtagtaaacacaaactccgcctcctccacaaccactccaaacactctctccctcaaacgcaaccgcagccgcagccgcaaccttcggcttcctcttcctcttcctcctcctcttcctcctccaaatccaccaaaccccgaaaaagcaagaacaagaacaacactaatctctctttgggtggtagtcaaatgatggggatgtttccaccggaaccggcgtttctcttcccggtctccactgtcggagggtttgaaggtatcaccgtctcatcccaattagggtttctctccggtgatatgattgagcaacaaaaaccggctccaacgtgtaccggactcctgctgagtgagatcatgaacggtagtgtgagttatggaactcatcatcaacaacacttgagtgagaaagaagttgaagaaatgaggatgaagatgttgcaacagccacagactcagatttgttacgctaccactaatcatcaaatagcttcttacaacaacaacaacaacaacaataacatcatgcttcatattcctcccactacttctactgccaccactattactacttcgcattctctcgctactgtcccatcaacttcggaccagcttcaagttcaagcggacgcacgaataagagttttcatcaatgaaatggagcttgaagtgagctcaggaccgttcaatgtgagggatgcatttggggaagaggttgttctgattaattccgcgggtcagcccattgtcaccgatgaatatggcgtcgctcttcaccctcttcaacacggagcctcgtactatctgatctagtcgtgtgggagatttgagtttgaagaagaaattaagacctgtctctttctttcaccatctctcgtacgtaggcttaaatgttaagattttataaagtattggtttcagttacctgttgtgacggtgtttatgtatgagtttcggacaacattcacaaaactctctcgttaaattgttgaccaataatatatgatgtgtgtttcattatt

SEQ ID NO: 7 - DNA, 1527 bp, Arabidopsis thaliana; identical to SEQ ID NO: 6
  misc_feature: AT4G17490

SEQ ID NO: 8 - DNA, 1092 bp, Arabidopsis thaliana
  misc_feature: AT5G43290
aaatagaaattcaacacacgttactcttaaaagttaaaagaataaagaatccctctctccctctccccctctccccctcaccaccagctcgcataagttttcttgtccccatactcatatggaagaagaaggttatcagtgggcaagaaggtgtggaaacaacgccgttgaagatccttttgtttatgagccacctcttttctttctgcctcaagaccaacatcatatgcatgggctcatgccaaatgaagatttcattgccaacaagttcgtcacatcaacactttactccggtccacgaatccaagatattgcaaacgctctggccttggtcgaacccctgacccacccagtccgagaaatctctaaatcaacagttcctcttttggaaagaagtactttgagcaaggtggataggtacactttaaaggtgaagaacaatagtaatggaatgtgtgatgatggatacaaatggagaaaatatggccaaaaatcaatcaaaaatagccctaacccaaggagttattacaagtgcacaaacccaatatgcaatgcaaagaagcaagtggagagatcaattgatgagtctaacacatatatcattacctacgaaggtttccacttccactatacttaccctttcttcctacccgataagacacgtcaatggcccaataaaaaaacgaaaatacataaacataatgctcaagatatgaacaaaaaatctcaaacccaagaagagagcaaagaagcacaattaggtgagctcaccaaccaaaatcatccagtcaataaagcccaagaaaacacaccggcaaatctcgaagagggattgttttttccggttgatcagtgtcggccgcaacaagggcttctagaagatgtggtagctccggcaatgaaaaatattcctaccagggacagtgttttgacagcttcttgataaataaaaaattatttgaaaatattaaaaaaatataaaattaaacttctttatcataaccggagaaagttttatttttcttctaaactttttacgtttaaataaatttattttgctcatctacacaataaatacatctaaagagtttc

SEQ ID NO: 9 - DNA, 645 bp, Arabidopsis thaliana
  misc_feature: AT2G45660
atggtgaggggcaaaactcagatgaagagaatagagaatgcaacaagcagacaagtgactttctccaaaagaaggaatggtttgttgaagaaagcctttgagctctcagtgctttgtgatgctgaagtttctcttatcatcttctctcctaaaggcaaactttatgaattcgccagctccaatatgcaagataccatagatcgttatctgaggcatactaaggatcgagtcagcaccaaaccggtttctgaagaaaatatgcagcatttgaaatatgaagcagcaaacatgatgaagaaaattgaacaactcgaagcttctaaacgtaaactcttgggagaaggcataggaacatgctcaatcgaggagctgcaacagattgagcaacagcttgagaaaagtgtcaaatgtattcgagcaagaaagactcaagtgtttaaggaacaaattgagcagctcaagcaaaaggagaaagctctagctgcagaaaacgagaagctctctgaaaagtggggatctcatgaaagcgaagtttggtcaaataagaatcaagaaagtactggaagaggtgatgaagagagtagcccaagttctgaagtagagacgcaattgttcattgggttaccttgttcttcaagaaagtga

SEQ ID NO: 10 - DNA, 1175 bp, Arabidopsis thaliana
  misc_feature: AT4G05100
acgtctctctctttctctctactctctgtttcctcataattcaatcactatatttttttaaaaacatttgacttcatcgatcggttaacaattaatcaaaaagatgggacgatcaccatgttgtgagaagaagaatggtctcaagaaaggaccatggactcctgaggaggatcaaaagctcattgattatatcaatatacatggttatggaaattggagaactcttcccaagaatgctgggttacaaagatgtggtaagagttgtcgtctccggtggaccaactatctccgaccagatattaagcgtggaagattctcttttgaagaagaagaaaccattattcaacttcacagcatcatgggaaacaagtggtctgcgattgcggctcgtttgcctggaagaacagacaacgagatcaaaaactattggaacactcacatcagaaaaagacttctaaagatgggaatcgacccggttacacacactccacgtcttgatcttctcgatatctcctccattctcagctcatctatctacaactcttcgcatcatcatcatcatcatcatcaacaacatatgaacatgtcgaggctcatgatgagtgatggtaatcatcaaccattggttaaccccgagatactcaaactcgcaacctctctcttttcaaaccaaaaccaccccaacaacacacacgagaacaacacggttaaccaaaccgaagtaaaccaataccaaaccggttacaacatgcctggtaatgaagaattacaatcttggttccctatcatggatcaattcacgaatttccaagacctcatgccaatgaagacgacggtccaaaattcattgtcatacgatgatgattgttcgaagtccaattttgtattagaaccttattactccgactttgcttcagtcttgaccacaccttcttcaagcccgactccgttaaactcaagttcctcaacttacatcaatagtagcacttgcagcaccgaggatgaaaaagagagttattacagtgataatatcactaattattcgtttgatgttaatggttttctccaattccaataaacaaaacgccattggaatagagttatgtaaacatgcaatcattgtatttgttatatagattttgttacatatccaaaatccaaaatactatagtttt

SEQ ID NO: 11 - DNA, 703 bp, Arabidopsis thaliana
  misc_feature: AT5G64810
aaatgaatatctctcaaaaccctagccctaattttacgtacttctccgatgaaaactttattaatccgtttatggataacaacgatttctcaaatttgatgttctttgacatagatgaaggaggtaacaatggattaatcgaggaagagatctcatctccgacaagcatcgtttcgtcggagacatttaccggggaaagcggcggatccggcagcgcaacaacgttgagtaaaaaggaatcaactaatagaggaagtaaagagagtgatcagacgaaggagacgggtcatcgagttgcatttagaacgagatcgaagattgatgtgatggatgatggttttaaatggaggaagtatggcaagaaatctgtcaaaaacaacattaacaagaggaattactacaaatgctcaagtgaaggttgctcggtgaagaagagggtagagagagatggtgacgatgcagcttatgtaattacaacatatgaaggagtccataaccatgagagtctctctaatgtctattacaatgaaatggttttatcttatgatcatgataactggaaccaacactctcttcttcgatcttaatccaaatctctcatcctcgttgagattgtaagataccaacaatattaatattataatctcaatgctttgtataatttgataggtgtataataaaggagttacttatttcgtggtta

SEQ ID NO: 12 - DNA, 962 bp, Arabidopsis thaliana
  misc_feature: AT5G13790
cttccttctaccttcttctctctgttcaattttgggggaaaatgggtcgtggaaaaatcgagataaagaggatcgagaatgcgaatagcagacaagtcactttttccaagaggcgttctgggttacttaagaaagctcgtgagctctctgttctttgtgatgctgaagttgctgtcatcgtcttctctaagtctggcaagctcttcgagtactccagtactggaatgaagcaaacactttccagatacggtaatcaccagagttcttcagcttctaaagcagaggaggattgtgcagaggtggatattttaaaggatcaactttcaaagcttcaagagaaacatttacaactgcagggcaagggcttgaatcctctgacctttaaagagctgcaaagccttgagcagcaactatatcatgcattgattactgtcagagagcgaaaggaacgattgctgactaaccaacttgaagaatcacgcctcaaggaacaacgagcagagttggaaaacgagaccttgcgtagacaggttcaagaactgaggagctttctcccgtcgttcacccactatgttccatcctacatcaaatgctttgctatagatccaaagaacgctctcataaaccacgacagtaaatgcagcctccagaacaccgattcagacacaactttgcaattagggttgccgggagaggcacatgatagaaggacgaatgaaggagaaagagagagcccgtcaagcgattcagtgacaacaaacacgagcagcgaaactgcagaaagaggggatcagtctagtttagcaaattctccacctgaagccaaaagacaaaggttctctgtttagtcctagaaaagtatgggagaaggctactaatgtttcctctttagcaagtatccgattgttttaaaagtaattttagagggatacttgcaaaaagaagagaagattcagttatct

SEQ ID NO: 13 - protein, 264 aa, Arabidopsis thaliana
  SITE (1)..(264): AT5G45580
MMTRDPKPRLRWTADLHDRFVDAVAKLGGADKATPKSVLKLMGLKGLTLYHLKSHLQKYRLGQQQGKKQNRTEQNKENAGSSYVHFDNCSQGGISNDSRFDNHQRQSGNVPFAEAMRHQVDAQQRFQEQLEVQKKLQMRMEAQGKYLLTLLEKAQKSLPCGNAGETDKGQFSDFNLALSGLVGSDRKNEKAGLVTDISHLNGGDSSQEFRLCGEQEKIETGDACVKPESGFVHFDLNSKSGYDLLNCGKYGIEVKPNVIGDRLQ

SEQ ID NO: 14 - protein, 359 aa, Arabidopsis thaliana
  SITE (1)..(359): AT1G67260
MSSSTNDYNDGNNNGVYPLSLYLSSLSGHQDIIHNPYNHQLKASPGHMVSAVPESLIDYMAFKSNNVVNQQGFEFPEVSKEIKKVVKKDRHSKIQTAQGIRDRRVRLSIGIARQFFDLQDMLGFDKASKTLDWLLKKSRKAIKEVVQAKNLNNDDEDFGNIGGDVEQEEEKEEDDNGDKSFVYGLSPGYGEEEVVCEATKAGIRKKKSELRNISSKGLGAKARGKAKERTKEMMAYDNPETASDITQSEIMDPFKRSIVFNEGEDMTHLFYKEPIEEFDNQESILTNMTLPTKMGQSYNQNNGILMLVDQSSSSNYNTFLPQNLDYSYDQNPFHDQTLYVVTDKNFPKGKVWIQDSFVN

SEQ ID NO: 15 - protein, 282 aa, Arabidopsis thaliana
  SITE (1)..(282): AT1G66230
MGRQPCCDKVGLKKGPWTAEEDRKLINFILTNGQCCWRAVPKLSGLLRCGKSCRLRWTNYLRPDLKRGLLSDYEEKMVIDLHSQLGNRWSKIASHLPGRTDNEIKNHWNTHIKKKLRKMGIDPLTHKPLSIVEKEDEEPLKKLQNNTVPFQETMERPLENNIKNISRLEESLGDDQFMEINLEYGVEDVPLIETESLDLICSNSTMSSSTSTSSHSSNDSSFLKDLQFPEFEWSDYGNSNNDNNNGVDNIIENNMMSLWEISDFSSLDLLLNDESSSTFGLF

SEQ ID NO: 16 - protein, 542 aa, Arabidopsis thaliana
  SITE (1)..(542): AT3G50650
MAYMCTDSGNLMAIAQQLIKQKQQQQSQHQQQEEQEQEPNPWPNPSFGFTLPGSGFSDPFQVTNDPGFHFPHLEHHQNAAVASEEFDSDEWMESLINGGDASQTNPDFPIYGHDPFVSFPSRLSAPSYLNRVNKDDSASQQLPPPPASTAIWSPSPPSPQHPPPPPPQPDFDLNQPIFKAIHDYARKPETKPDTLIRIKESVSESGDPIQRVGYYFAEALSHKETESPSSSSSSSLEDFILSYKTLNDACPYSKFAHLTANQAILEATNQSNNIHIVDFGIFQGIQWSALLQALATRSSGKPTRIRISGIPAPSLGDSPGPSLIATGNRLRDFAAILDLNFEFYPVLTPIQLLNGSSFRVDPDEVLVVNFMLELYKLLDETATTVGTALRLARSLNPRIVTLGEYEVSLNRVEFANRVKNSLRFYSAVFESLEPNLDRDSKERLRVERVLFGRRIMDLVRSDDDNNKPGTRFGLMEEKEQWRVLMEKAGFEPVKPSNYAVSQAKLLLWNYNYSTLYSLVESEPGFISLAWNNVPLLTVSSWR

SEQ ID NO: 17 - protein, 218 aa, Arabidopsis thaliana
  SITE (1)..(218): AT3G23240
MDPFLIQSPFSGFSPEYSIGSSPDSFSSSSSNNYSLPFNENDSEEMFLYGLIEQSTQQTYIDSDSQDLPIKSVSSRKSEKSYRGVRRRPWGKFAAEIRDSTRNGIRVWLGTFESAEEAALAYDQAAFSMRGSSAILNFSAERVQESLSEIKYTYEDGCSPVVALKRKHSMRRRMTNKKTKDSDFDHRSVKLDNVVVFEDLGEQYLEELLGSSENSGTW

SEQ ID NO: 18 - protein, 378 aa, Arabidopsis thaliana
  SITE (1)..(378): AT2G33880
MASSNRHWPSMFKSKPHPHQWQHDINSPLLPSASHRSSPFSSGCEVERSPEPKPRWNPKPEQIRILEAIFNSGMVNPPREEIRRIRAQLQEYGQVGDANVFYWFQNRKSRSKHKLRLLHNHSKHSLPQTQPQPQPQPSASSSSSSSSSSSKSTKPRKSKNKNNTNLSLGGSQMMGMFPPEPAFLFPVSTVGGFEGITVSSQLGFLSGDMIEQQKPAPTCTGLLLSEIMNGSVSYGTHHQQHLSEKEVEEMRMKMLQQPQTQICYATTNHQIASYNNNNNNNNIMLHIPPTTSTATTITTSHSLATVPSTSDQLQVQADARIRVFINEMELEVSSGPFNVRDAFGEEVVLINSAGQPIVTDEYGVALHPLQHGASYYLI

SEQ ID NO: 19 - protein, 282 aa, Arabidopsis thaliana
  SITE (1)..(282): AT4G17490
MATPNEVSALFLIKKYLLDELSPLPTTATTNRWMNDFTSFDQTGFEFSEFETKPEIIDLVTPKPEIFDFDVKSEIPSESNDSFTFQSNPPRVTVQSNRKPPLKIAPPNRTKWIQFATGNPKPELPVPVVAAEEKRHYRGVRMRPWGKFAAEIRDPTRRGTRVWLGTFETAIEAARAYDKEAFRLRGSKAILNFPLEVDKWNPRAEDGRGLYNKRKRDGEEEEVTVVEKVLKTEESYDVSGGENVESGLTAIDDWDLTEFLSMPLLSPLSPHPPFGYPQLTVV

SEQ ID NO: 20 - protein, 274 aa, Arabidopsis thaliana
  SITE (1)..(274): AT5G43290
MEEEGYQWARRCGNNAVEDPFVYEPPLFFLPQDQHHMHGLMPNEDFIANKFVTSTLYSGPRIQDIANALALVEPLTHPVREISKSTVPLLERSTLSKVDRYTLKVKNNSNGMCDDGYKWRKYGQKSIKNSPNPRSYYKCTNPICNAKKQVERSIDESNTYIITYEGFHFHYTYPFFLPDKTRQWPNKKTKIHKHNAQDMNKKSQTQEESKEAQLGELTNQNHPVNKAQENTPANLEEGLFFPVDQCRPQQGLLEDVVAPAMKNIPTRDSVLTAS

SEQ ID NO: 21 - protein, 214 aa, Arabidopsis thaliana
  SITE (1)..(214): AT2G45660
MVRGKTQMKRIENATSRQVTFSKRRNGLLKKAFELSVLCDAEVSLIIFSPKGKLYEFASSNMQDTIDRYLRHTKDRVSTKPVSEENMQHLKYEAANMMKKIEQLEASKRKLLGEGIGTCSIEELQQIEQQLEKSVKCIRARKTQVFKEQIEQLKQKEKALAAENEKLSEKWGSHESEVWSNKNQESTGRGDEESSPSSEVETQLFIGLPCSSRK

SEQ ID NO: 22 - protein, 324 aa, Arabidopsis thaliana
  SITE (1)..(324): AT4G05100
MGRSPCCEKKNGLKKGPWTPEEDQKLIDYINIHGYGNWRTLPKNAGLQRCGKSCRLRWTNYLRPDIKRGRFSFEEEETIIQLHSIMGNKWSAIAARLPGRTDNEIKNYWNTHIRKRLLKMGIDPVTHTPRLDLLDISSILSSSIYNSSHHHHHHHQQHMNMSRLMMSDGNHQPLVNPEILKLATSLFSNQNHPNNTHENNTVNQTEVNQYQTGYNMPGNEELQSWFPIMDQFTNFQDLMPMKTTVQNSLSYDDDCSKSNFVLEPYYSDFASVLTTPSSSPTPLNSSSSTYINSSTCSTEDEKESYYSDNITNYSFDVNGFLQFQ

SEQ ID NO: 23 - protein, 194 aa, Arabidopsis thaliana
  SITE (1)..(194): AT5G64810
MNISQNPSPNFTYFSDENFINPFMDNNDFSNLMFFDIDEGGNNGLIEEEISSPTSIVSSETFTGESGGSGSATTLSKKESTNRGSKESDQTKETGHRVAFRTRSKIDVMDDGFKWRKYGKKSVKNNINKRNYYKCSSEGCSVKKRVERDGDDAAYVITTYEGVHNHESLSNVYYNEMVLSYDHDNWNQHSLLRS

SEQ ID NO: 24 - protein, 268 aa, Arabidopsis thaliana
  SITE (1)..(268): AT5G13790
MGRGKIEIKRIENANSRQVTFSKRRSGLLKKARELSVLCDAEVAVIVFSKSGKLFEYSSTGMKQTLSRYGNHQSSSASKAEEDCAEVDILKDQLSKLQEKHLQLQGKGLNPLTFKELQSLEQQLYHALITVRERKERLLTNQLEESRLKEQRAELENETLRRQVQELRSFLPSFTHYVPSYIKCFAIDPKNALINHDSKCSLQNTDSDTTLQLGLPGEAHDRRTNEGERESPSSDSVTTNTSSETAERGDQSSLANSPPEAKRQRFSV

SEQ ID NO: 25 - protein, 542 aa, Arabidopsis thaliana
  SITE (1)..(542): AT3G50650_mod
MAYMCTDSGNLMAIAQQLIKQKQQQQSQHQQQEEQEQEPNPWPNPSFGFTLPGSGFSDPFQVTNDPGFHFPHLEHHQNAAVASEEFDSDEWMESLINGGDASQTNPDFPIYGHDPFVSFPSRLSAPSYLNRVNKDDSASQQLPPPPASTAIWSPSPPSPQHPPPPPPQPDFDLNQPIFKAIHDYARKPETKPDTLIRIKESVSESGDPIQRVGYYFAEALSHKETESPSSSTSSSLEDFILSYKTLNDACPYSKFAHLTANQAILEATNQSNNIHIVDFGIFQGIQWSALLQALATRSSGKPTRIRISGIPAPSLGDSPGPSLIATGNRLRDFAAILDLNFEFYPVLTPIQLLNGSSFRVDPDEVLVVNFMLELYKLLDETATTVGTALRLARSLNPRIVTLGEYEVSLNRVEFANRVKNSLRFYSAVFESLEPNLDRDSKERLRVERVLFGRRIMDLVRSDDDNNKPGTRFGLMEEKEQWRVLMEKAGFEPVKPSNYAVSQAKLLLWNYNYSTLYSLVESEPGFISLAWNNVPLLTVSSWR

SEQ ID NO: 26 - protein, 378 aa, Arabidopsis thaliana
  SITE (1)..(378): AT2G33880_mod
MASSNRHWPSMFKSKPHPHQWQHDINSPLLPSASHRSSPFSSGCEVERSPEPKPRWNPKPEQIRILEAIFNSGMVNPPREEIRRIRAQLQEYGQVGDANVFYWFQNRKSRSKHKLRLLHNHSKHSLPQTQPQPQPQPSASSSSTSSSSSSKSTKPRKSKNKNNTNLSLGGSQMMGMFPPEPAFLFPVSTVGGFEGITVSSQLGFLSGDMIEQQKPAPTCTGLLLSEIMNGSVSYGTHHQQHLSEKEVEEMRMKMLQQPQTQICYATTNHQIASYNNNNNNNNIMLHIPPTTSTATTITTSHSLATVPSTSDQLQVQADARIRVFINEMELEVSSGPFNVRDAFGEEVVLINSAGQPIVTDEYGVALHPLQHGASYYLI

SEQ ID NO: 27 - protein, 214 aa, Arabidopsis thaliana
  SITE (1)..(214): AT2G45660_mod
MVRGKTQMKRIENATSRQVTFSKRRNGLLKKAFELSVLCDAEVSLIIFSPKGKLYEFASSNMQDTIDRYLRHTKDRVSTKPVSEENMQHLKYEAANMMKKIEQLEASKRKLLGEGIGTCSIDELQQIEQQLEKSVKCIRARKTQVFKEQIEQLKQKEKALAAENEKLSEKWGSHESEVWSNKNQESTGRGDEESSPSSEVETQLFIGLPCSSRK

SEQ ID NO: 28 - DNA, 1413 bp, Oryza sativa
  misc_feature: actin promoter and first intron
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccacc

SEQ ID NO: 29 - DNA, 512 bp, Arabidopsis thaliana
  misc_feature: Sac66 terminator
aacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 30 - DNA, 2720 bp, Artificial
  pBIOS03693     Actin-AtG2_like-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatgatgacacgggaccccaagcctcgcctgcgctggactgctgatctgcatgataggttcgttgacgctgttgccaagctcggcggcgctgacaaggccaccccgaagtcggttctcaagctgatgggcctcaaggggctcacgctgtaccacctcaagtctcatctgcagaagtacaggctgggccagcagcaggggaagaagcagaaccggaccgagcagaacaaggagaatgccggctccagctacgtccacttcgacaattgctcgcagggcgggatcagcaacgactcgcgcttcgataatcaccagaggcagtccggcaacgtcccattcgccgaggcgatgcggcatcaggttgatgctcagcagcgcttccaggagcagctggaggtgcagaagaagctgcagatgcggatggaggcgcagggcaagtacctcctgacactcctggagaaggcgcagaagtccctcccttgcggcaacgctggggagactgacaagggccagttcagcgatttcaatctcgctctgtctggcctggtggggtcagaccgcaagaacgagaaggcgggcctcgtcaccgacatcagccacctgaatggcggggattcgtctcaggagttcaggctctgcggcgagcaggagaagattgagacgggggatgcgtgcgttaagccggagtccggcttcgtgcatttcgacctgaactcaaagtccgggtacgatctcctgaattgcggcaagtacgggatcgaggtgaagcccaacgtcattggcgacaggctccagtgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 31 - DNA, 3005 bp, Artificial
  pBIOS03694     Actin-AtTCP1-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatgtcgtcctccaccaatgattacaatgatggcaacaacaatggcgtttacccactctcgctctacctgtcgtcgctgtcggggcaccaggacatcattcacaacccgtacaatcatcagctcaaggcttctccaggccacatggtcagcgccgttcccgagtcgctgatcgattacatggcgttcaagtcaaacaatgtggtcaaccagcagggcttcgagttccccgaggtgtcaaaggagatcaagaaggttgtgaagaaggaccggcattccaagatccagacggctcagggcattagggataggagggtccgcctctccatcgggattgctcgccagttcttcgacctccaggatatgctgggcttcgacaaggcctcgaagacactcgattggctcctgaagaagtctaggaaggcgatcaaggaggtcgttcaggctaagaacctgaacaatgacgatgaggacttcggcaatattggcggggatgttgagcaggaggaggagaaggaggaggatgataacggcgacaagtccttcgtctacgggctgagcccaggctacggggaggaggaggtggtctgcgaggctactaaggccggcatccgcaagaagaagtccgagctcaggaacatttccagcaagggcctgggggctaaggcgagggggaaggccaaggagcggaccaaggagatgatggcctacgacaacccggagactgcgtctgatatcacccagtcagagattatggaccccttcaagcggtccatcgtgttcaacgagggcgaggatatgacccacctcttctacaaggagccaatcgaggagttcgacaaccaggagagcattctcaccaatatgacgctgcctacaaagatgggccagtcgtacaaccagaacaatgggatcctcatgctggtcgaccagtcgtcttcatccaactacaataccttcctcccacagaacctggactactcctacgatcagaacccgtttcatgaccagacgctctacgtggtgacagataagaacttcccaaagggcaaggtctggatccaggacagcttcgtcaattgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 32 - DNA, 2774 bp, Artificial
  pBIOS03695     Actin-AtMYB20-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggggcgccagccatgctgcgacaaggttgggctcaagaaggggccgtggactgccgaggaggacaggaagctcatcaatttcatcctgaccaacggccagtgctgctggagggccgtcccgaagctgtccggcctcctgcgctgcggcaagtcctgcaggctcaggtggacgaactacctgaggccagatctcaagagggggctcctgtcggactacgaggagaagatggttatcgatctgcactcccagctcggcaatcgctggagcaagattgcgtcgcatctgccggggaggaccgacaacgagatcaagaatcactggaacacgcacatcaagaagaagctcaggaagatgggcatcgacccgctgacacacaagcccctctccattgttgagaaggaggatgaggagcccctgaagaagctccagaacaatacagtgccattccaggagactatggagcggcctctggagaacaatatcaagaacatttctcgcctggaggagtcactcggcgacgatcagttcatggagatcaacctggagtacggggtggaggacgtcccactcatcgagacagagagcctggatctcatttgctcaaattccactatgtccagctcgacctccacgtcttcacattccagcaacgattcgtctttcctgaaggacctccagttccctgagttcgagtggtccgactacggcaatagcaacaatgataacaataacggggtggacaacatcattgagaataacatgatgtcgctgtgggagatctctgacttctcatccctcgatctcctgctcaacgacgagagctcgtctaccttcggcctcttctgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 33 - DNA, 3554 bp, Artificial
  pBIOS03696     Actin AtSCL7-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggcgtacatgtgcacggacagcgggaacctgatggctattgcccagcagctcattaagcagaagcagcagcagcagtcgcagcatcagcagcaggaggagcaggagcaggagccaaacccctggccaaatccttccttcggcttcaccctgccaggctcagggttctccgatcctttccaggttacgaacgacccggggttccacttcccccacctggagcaccatcagaatgccgcggtcgccagcgaggagttcgattcggacgagtggatggagtccctgatcaacggcggggatgcgagccagacaaatccggacttccccatctacggccacgatccattcgtcagcttcccttcgaggctctctgcgccgtcatacctgaaccgggttaataaggacgattcggcttctcagcagctcccaccaccacctgcttcgacggctatctggtcaccatccccaccatctccacagcacccacctccacccccacctcagccggatttcgacctcaaccagcccatcttcaaggcgattcatgactacgctcggaagccggagacaaagcccgatactctgatccgcattaaggagagcgtgtcggagtctggcgacccaatccagagggtcgggtactacttcgcggaggctctgtctcacaaggagacagagtcaccctccagctcgacttcttcatccctggaggacttcatcctctcctacaagaccctgaacgatgcctgcccctacagcaagttcgcgcacctcacagcgaaccaggctattctggaggccactaatcagtccaacaatatccatattgtggacttcggcatcttccaggggattcagtggagcgcgctcctgcaggccctggcgaccaggagctcgggcaagccaacgaggatcaggattagcgggatcccagctccatccctcggcgacagcccagggccttcgctcatcgcgacaggcaacaggctgcgggatttcgctgccattctggacctcaatttcgagttctacccagtcctgactcctattcagctcctgaacggctcctccttccgcgtcgatccggacgaggttctcgtggtcaacttcatgctggagctctacaagctcctggacgagaccgctaccacggtgggcacggccctgcggctcgcgaggtcgctcaacccaaggatcgtcaccctgggggagtacgaggtgtccctcaatagggtcgagttcgctaaccgggttaagaattctctccgcttctactcagccgtgttcgagtccctggagccaaacctcgatcgcgactccaaggagcgcctcagggttgagagggtgctgttcggccgcaggatcatggacctggtgaggtccgacgatgacaacaataagccaggcaccaggttcgggctgatggaggagaaggagcagtggcgcgtcctcatggagaaggccggcttcgagccagtcaagcctagcaactacgctgtttcgcaggccaagctcctgctctggaactacaattactctaccctgtactcactcgtggagtccgagccaggcttcatctccctcgcgtggaacaatgttcctctgctcacggtgtccagctggcgctgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 34 - DNA, 2582 bp, Artificial
  pBIOS03697     Actin-AtERF1-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggaccctttcctcattcagtcacccttctctggcttctcccccgagtacagcattggctcatcacccgattcgttctcctcttcctccagcaacaattacagcctcccattcaacgagaatgattcggaggagatgttcctctacggcctgatcgagcagtccacccagcagacgtacattgactctgattcacaggacctgccgatcaagagcgtttcgtctcgcaagtcggagaagtcttacaggggcgtgcgcaggaggccctgggggaagttcgccgcggagatccgcgattcgaccaggaacggcattcgggtctggctcgggacgttcgagtctgctgaggaggctgccctggcgtacgaccaggctgctttctccatgcggggctcatccgctattctcaatttcagcgctgagcgcgttcaggagtccctgagcgagatcaagtacacatacgaggacgggtgctctccagtggtcgctctcaagaggaagcactcaatgcgcaggcggatgacaaacaagaagactaaggactcagatttcgaccatcggtccgtcaagctggataatgttgtggtcttcgaggacctcggcgagcagtacctggaggagctcctgggcagctcggagaactccgggacgtggtgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 35 - DNA, 3062 bp, Artificial
  pBIOS03698     Actin-AtHB3-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggcgtcctcaaatcggcattggccctctatgttcaagtctaagccgcaccctcatcagtggcagcacgacatcaactcccctctgctgccttctgcttcacacaggtccagcccgttctcgtctggctgcgaggttgagcgctccccagagcctaagccgaggtggaaccccaagccagagcagatccggattctggaggcgatcttcaacagcggcatggtcaatccaccccgggaggagatccgcaggattagggctcagctccaggagtacggccaggtcggggacgccaacgttttctactggttccagaataggaagtcccggagcaagcacaagctgcgcctcctgcacaaccattctaagcattcactccctcagacacagcctcagccacagccacagccatccgcctcatccagctcgacatcttcatccagctcgtctaagtcgactaagccgaggaagtctaagaacaagaacaatacgaatctgtccctcggcgggagccagatgatgggcatgttcccacctgagcccgccttcctcttcccagtttcaaccgtgggcgggttcgagggcatcacggtgtcatcccagctgggcttcctctctggggatatgattgagcagcagaagcctgctccaacctgcacgggcctcctgctctccgagatcatgaacggctcggtgtcttacgggacccaccatcagcagcacctgagcgagaaggaggtcgaggagatgcggatgaagatgctccagcagccgcagacgcagatctgctacgctaccacgaaccaccagattgcctcgtacaacaataacaataacaataacaatataatgctgcacatcccgcccacaacttccaccgccaccacgatcacaacttcacattccctggcgacagtccccagcacttcggaccagctccaggtccaggctgatgctaggatccgcgttttcattaacgagatggagctggaggttagctcgggcccattcaatgtgagggacgcgttcggggaggaggtggtcctcatcaacagcgctggccagcccattgtgaccgatgagtacggggtcgcgctgcacccactccagcatggcgcttcgtactacctcatctgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 36 - DNA, 2774 bp, Artificial
  pBIOS03699     Actin-AtERF6-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggctacccccaacgaggtttccgccctgttcctgattaagaagtacctgctggatgagctgtccccactcccgactaccgctaccacgaaccgctggatgaacgactttacctctttcgatcagacgggcttcgagttctcagagttcgagaccaagccggagatcattgacctcgtgacgccgaagcccgagatcttcgacttcgatgtcaagtcggagattccatccgagagcaacgatagcttcacattccagtcgaatccaccccgcgtgaccgtccagagcaacaggaagccacctctcaagatcgctccgcccaataggacaaagtggattcagttcgccactggcaacccaaagcctgagctgccggttcccgtggtcgctgctgaggagaagaggcactacaggggcgtccggatgaggccgtgggggaagttcgctgctgagatcagggaccctacaaggaggggcactcgcgtgtggctcgggaccttcgagacggctattgaggctgctcgggcctacgacaaggaggcgttcaggctgaggggctccaaggcgatcctcaacttcccgctggaggtcgacaagtggaatcccagggctgaggatggcagggggctgtacaataagcgcaagagggacggcgaggaggaggaggttaccgttgtggagaaggtgctcaagacggaggagtcatacgacgtctccggcggggagaacgttgagtccggcctgacagcgatcgacgattgggatctcactgagttcctgagcatgccgctcctgtcgccactctctcctcatccacctttcggctacccgcagctgaccgtcgtttgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 37 - DNA, 2750 bp, Artificial
  pBIOS03700     Actin-AtWRKY49-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcatcccccctccccctccgccgccgccgcgccggtaaccaccccgcccctatcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggaggaggagggctaccagtgggcgaggcgctgcgggaataacgctgttgaggaccccttcgtctacgagccacccctgttcttcctcccgcaggaccagcaccatatgcacggcctgatgcccaacgaggatttcatcgcgaataagttcgttacctcaacgctctactccgggccaaggatccaggacattgccaacgcgctcgctctggttgagccactgacgcatcctgtgcgggagatttccaagagcacagtcccgctcctggagcgctcgactctctctaaggtcgataggtacaccctgaaggttaagaacaattccaacggcatgtgcgacgatgggtacaagtggcggaagtacggccagaagtccatcaagaactcgccgaatccccgctcctactacaagtgcacaaaccccatctgcaatgccaagaagcaggtggagcggtctattgacgagtcaaacacatacatcattacttacgaggggttccacttccattacacctacccgttcttcctccccgacaagacgaggcagtggccgaataagaagacaaagatccacaagcataacgcgcaggatatgaataagaagtcgcagactcaggaggagagcaaggaggctcagctcggcgagctgaccaaccagaatcacccagtgaacaaggctcaggagaacacgcctgccaacctggaggagggcctgttcttcccagtcgaccagtgcaggcctcagcaggggctcctggaggatgtggtcgctccagcgatgaagaacatccccaccagggactccgtcctgacggcgagctgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 38 - DNA, 2570 bp, Artificial
  pBIOS03729     Actin-AtAGL20-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcatcccccctccccctccgccgccgccgcgccggtaaccaccccgcccctatcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggtcaggggcaagacgcagatgaagcggattgagaacgcgacaagcaggcaggttactttctccaagcggcggaatgggctcctcaagaaggcctttgagctctccgttctgtgcgacgctgaggtgtccctgatcattttcagcccaaagggcaagctctacgagttcgcttccagcaacatgcaggacaccatcgatcgctacctccgccacaccaaggatcgcgtgtcgacgaagccggtctctgaggagaacatgcagcatctgaagtacgaggccgcgaatatgatgaagaagattgagcagctggaggcgagcaagaggaagctcctgggcgaggggatcggcacctgctcgattgacgagctccagcagatcgagcagcagctggagaagtccgtcaagtgcattcgcgccaggaagacgcaggttttcaaggagcagatcgagcagctcaagcagaaggagaaggcgctggctgccgagaatgagaagctctctgagaagtgggggtctcacgagtcagaagtgtggtcaaacaagaatcaggagtccacagggaggggcgatgaggagtcgtctccgtcatccgaggtcgagactcagctgttcatcggcctgccctgcagctcgcggaagtgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 39 - DNA, 2900 bp, Artificial
  pBIOS03730     Actin-AtMYB74-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcatcccccctccccctccgccgccgccgcgccggtaaccaccccgcccctatcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggggaggtcaccgtgctgcgagaagaagaatgggctcaagaaggggccgtggacacccgaggaggaccagaagctgattgattacatcaacattcacggctacgggaattggcggaccctcccaaagaacgctggcctgcagcgctgcgggaagtcctgcaggctcaggtggacgaactacctgcgccctgacatcaagcggggccgcttctccttcgaggaggaggagacaatcattcagctccactctatcatgggcaacaagtggagcgctattgccgcgcgcctgccagggaggaccgacaacgagatcaagaactactggaatacgcatattaggaagcggctcctgaagatgggcatcgatccggtcacccacacgcccaggctcgatctcctggacatctccagcattctgtcgtcttcaatctacaactccagccaccatcaccatcaccatcaccagcagcatatgaatatgagccggctcatgatgtcggacgggaaccaccagccactggttaatcctgagatcctcaagctggcgacatccctcttcagcaaccagaatcatccgaacaatactcacgagaacaataccgtgaaccagacggaggtcaatcagtaccagacgggctacaacatgccagggaatgaggagctgcagagctggttccctatcatggatcagttcaccaacttccaggacctcatgcccatgaagaccacggtgcagaacagcctgtcgtacgacgatgactgctctaagtcaaatttcgtgctggagccgtactactcagacttcgcttccgtgctcacaactccctcgtcttcaccgacacccctgaactccagctcgtctacttacatcaattcatccacatgctcgactgaggatgagaaggagtcgtactactctgacaacattaccaattacagcttcgatgtcaacggcttcctgcagttccagtgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 40 - DNA, 2510 bp, Artificial
  pBIOS03731     Actin-AtWRKY51-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcatcccccctccccctccgccgccgccgcgccggtaaccaccccgcccctatcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatgaacatcagccagaatccctcccccaacttcacctacttcagcgacgagaacttcatcaatcccttcatggacaataacgacttctcgaacctcatgttcttcgacatcgatgagggcgggaacaatggcctgatcgaggaggagatttccagcccgaccagcattgtttcgtctgagaccttcacgggcgagtcgggcgggagcgggtcggctaccacgctctcgaagaaggagtctaccaacaggggctccaaggagagcgaccagacaaaggagactgggcacagggtggcgttccgcacgaggtctaagatcgatgtcatggacgatggcttcaagtggcgcaagtacgggaagaagtccgtcaagaacaatattaacaagaggaactactacaagtgctcatccgagggctgctccgtgaagaagcgggtcgagagggacggcgacgatgctgcctacgtgatcacaacttacgagggcgttcacaaccatgagtctctctcaaacgtttactacaatgagatggtgctgagctacgaccacgataactggaatcagcattcactcctgcggtcctgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 41 - DNA, 341 bp, Artificial
  pBIOS03732     Actin-AtAGL15-Sac66
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgcta

SEQ ID NO: 42 - protein, 271 aa, Zea mays
  SITE (1)..(271): GRMZM2G125704_P02 ZmG2 Like corn orthologues preferred
MQGSYGYDGAASRDPKPRLRWTPDLHQRFVDAVTKLGGPDRATPKSVLRLMGMKDLTLYQLKSHLQKYRLGIQGKKSTGLEPASGGVLRSQGFGSTTAHPPPGVPDQGKNTREIALSDALRYQIQVQRKLQEQTEVQKKLQMRIEAQGKYLKTILEKAQTNISFHTNASNGIESTRSQLMDFNLDGFMNNATQVCKEHREQLVKAMSDENDKDSLGLQLYHLGSQEAKEVKCTPKTEDSLLLDLNIKGGYDLSSRGMQACELELKINQQIV

SEQ ID NO: 43 - protein, 376 aa, Zea mays
  SITE (1)..(376): AC233950.1_FGP002
MFPFCDSSSPMDLPLYQQLQLSPSSPKTDQSSSFYCYPCSPPFAAADASFPLSYQIGSAAAADATPPQAVINSPDLPVQALMDHAPAPATELGACASGAEGSGASLDRAAAAARKDRHSKICTAGGMRDRRMRLSLDVARKFFALQDMLGFDKASKTVQWLLNTSKSAIQEIMADDASSECVEDGSSSLSVDGKHNPAEQLGGGGDQKPKGNCRGEGKKPAKASKAAATPKPPRKSANNAHQVPDKETRAKARERARERTKEKHRMRWVKLASAIDVEAAAASGPSDRPSSNNLSHHSSLSMNMPCAAAELEERERCSSALSNRSAGRMQEITGASDVVLGFGNGGGGYGDGGGNYYCQEQWELGGVVFQQNSRFY

SEQ ID NO: 44 - protein, 278 aa, Zea mays
  SITE (1)..(278): GRMZM2G048910_P01 ZmMYB 20  corn orthologues preferred
MGRQPCCDKVGVKKGPWTAEEDQKLVGFLLTHGHCCWRVVPKLAGLLRCGKSCRLRWTNYLRPDLKRGLLSDDEERLVIDLHAQLGNRWSKIAAQLPGRTDNEIKNHWNTHIRKKLVRMGIDPVTHLPLQEPPAPAPAPAEQQEQSHRQQEELQLQELQNGRELIMQEGAGEDDITPMIQPHEIMPTAAAASNCGSVSSAHAGSASVVSPSCSSSAVSGVEWPEPMYLLGMDGIMDADWGSLFPDTGAGGGGGGFDLGVDPFDQYPGGGFDQEDDHRI

SEQ ID NO: 45 - protein, 569 aa, Zea mays
  SITE (1)..(569): GRMZM2G104342_P01
MAYMCADSGNLMAIAQQVIQQQQQQQQQEHQRHHHHHLPMPLPLPPPPRQALPMPPATAPPHGQIPAASLPYGGGAWPQADHFFPDAFVGTSAADAVFSDLAAAADFDSDVWMNSLIGDAPVFADSDLERLIFTTPPPPPPVPAPAPAPASAVAPVDAAAQPEAATPASLPQPAAVAAPAACSSPSSLDAACSAPILQSLLACSRTAAAGTGLAAAELAEVRAAASDDGDPAERVAFYFADALARRLACGGGAQAQPSLAVDSRFAPDELTLCYKTLNDACPYSKFAHLTANQAILEATGAATKIHIVDFGIVQGIQWAALLQALATRPGEKPSRVRISGVPSPYLGPKPAASLAATSARLRDFAKLLGVDFEFVPLLRPVHELDRSDFLVEPDETVAVNFMLQLYHLLGDSDEPVRRVLRLVKSLDPSVVTLGEYEVSLNRAGFVDRFANALLYYKPVFESLDVAMPRDSPERVRVERCMFGERIRRAIGPEEGAERTDRMAASREWQTLMEWCGFEPVKLSNYAMSQADLLLWNYDSKYKYSLVELPPAFLSLAWEKQPLLTVSAWR

SEQ ID NO: 46 - protein, 262 aa, Zea mays
  SITE (1)..(262): AC233933.1_FGP001
MPSILAIKKVNQLQPPFLAPMGHPSSIHSFYLYSEYMATAAGAATSSSSGSSSDSLPFSMVCGDEAPAAVSWRAADAEVSVAAVPGAGLRQAPSKGAFIGVRRRPWGRFAAEIRDSTRNGARVWLGTFDSAEAAAMAYDQAALSARGSAAALNFPVERVQESLRALALGGNAAGASASAGGSPVLALKSRHSKRKRRKKSELLAAAAAKGAAAAAKATAAGGRSQIKNAAAAAEQQRFVVELEDLGAEYLEELLRISESYSS

SEQ ID NO: 47 - protein, 506 aa, Zea mays
  SITE (1)..(506): GRMZM2G409881_P01
MASSNRHWPSMYRSSLACNFQQPQPQPDMNNGGKSSLMSSRCEENGGRNPEPRPRWNPRPEQIRILEGIFNSGMVNPPRDEIRRIRLQLQEYGPVGDANVFYWFQNRKSRTKHKLRAAGQLQPSGSGRSALQARACAPAPVTPPRNLQLAAAAPVAPPTSSSSSSSDRSSGSSSSKSVTVTPTTAVALASPAGAAPAAVFRQQGVMPTTAMDLLTPLPSSSAALAARQLYYQYHSQIMAPAAPPMPDTVIASPEQFLPQWQQGGQQHYYLPATELGGVLDGHSHHTHEPPAAIHRPVSLSPSVLFGLCNEALRQDYCADISVVPTKGLGHGHQFWNSTTCGSDMGNSNSKIDAVSAVIRDDEKSRLGLLHYYGLAGATTTAAAAVAPAPLAADAAAGTATLLPSSAASDQLQGLLDAAGLLMGETPPTPTATVVAVARDAVTCAATATAQFSVPASMRLDVRLAFGEAALLARHTGEAVPVDESGVTVEPLQQDTLYYVLMQATNN

SEQ ID NO: 48 - protein, 315 aa, Zea mays
  SITE (1)..(315): GRMZM5G805505_P01
MDMDFNGDADSFALDFIRDLMLGGDSSIPVPSPVAASSDDVTCPVLPPQLEFQSVSFLPHQQRQGYIDLTTSECVGAAPAAATVVGEAVFRAQEPAPPVMIKFGTEPSSPLATTRQPLTISVPPSSYAWAAVEDYRKYRGVRQRPWGKYAAEIRDPKRRGSRAWLGTYDTPVEAARAYDRAAFRMRGAKAILNFPNEVGTRGADLWAAPTANKRKRQQMVEGEDDPEPEVEVVPMVNKAVKTEAQSSMTTQASSPSPSSTVASTTTGTTGGAGWLPVTPSSGSSEQYWETLLRGLPPLSPLSPHPALGFPLLSVN

SEQ ID NO: 49 - protein, 337 aa, Zea mays
  SITE (1)..(337): AC165171.2_FGP002
MEEEEGSSTSSVAVPVPPPPNKYDSWTTDPEELMMMMVDEAAVQQEGPRRRRDSMLNKLISTVYSGPTISDIESALSFTGGGDVDARNYNNSSAGPGALSSPEKVLMSKMENKYTLKIKTCGNGSSLAEDGYKWRKYGQKSIKNSPNPRSYYRCTNPRCNAKKQVERSTDEPDTLVVTYEGLHLHYTYSHFLHHQQPPPQPKKPKLGAGPPQQPIVVMDDDLLHGPAQQDIITTGPLGAAVMAPAPAPPPAAASFCYDLDDDVPAFFDDHDHHHHQQQHITNGGLLEDMVPLLVRRPCTTTTTTSAGSTSPDLSASSSVSWNPTSPYIDMAILSNIF

SEQ ID NO: 50 - protein, 224 aa, Zea mays
  SITE (1)..(224): GRMZM2G070034_P01 ZmAGL20  corn orthologues preferred
MVRGKTELKRIENATSRQVTFSKRRNGLLKKAFELSVLCDAEVGLVVFSPRGKLYEFASAASLQKTIDRYRTYTRENVNNKTVQQDIQQVKADAVSLASRLEALEKTKRMFLGENLEECSIEELHNLEVKLAKSLHVIRGKKTQLLEQQISKLKEKERTLLQDNKELRDKQRNLQSPPEAPPDLNRCVPPWPRSLPAPSNDMDVETELYIGLPGRERSSNRDSD

SEQ ID NO: 51 - protein, 262 aa, Zea mays
  SITE (1)..(262): GRMZM2G031323_P01
MPSILAIKKVNQLQPPFLAPMGHPSSIHSFYLYSEYMATAAAAATSSSSSSSSDSLPFSMVCGDEAPAAVSWRAADAEVSVAAVPGAGLRQAPSKGAFIGVRRRPWGRFAAEIRDSTRNGARVWLGTFDSAEAAAMAYDQAALSARGSAAALNFPVERVQESLRALALGGNAAGASASAGGSPVLALKSRHSKRKRRKKSELLAAAAAKGAAAAAKATAAGGRSQIKNAAAAAEQQRFVVELEDLGAEYLEELLRISESYSS

SEQ ID NO: 52 - protein, 213 aa, Zea mays
  SITE (1)..(213): GRMZM2G101405_P01
MHMALSSRSSFAADVLLPATMSYRQPCSGASSYLGSQPAAPFPSAAFGAVAQLDVFDCLSSDEGVGVPAAVPGAFAPPPPLMPAERVVPDAAAGYSSHTRSAAAVAGEGSRTTHRIAFRVRSDEDEVLDDGYKWRKYGKKSVKNSPNPRNYYRCSTEGCNVKKRVERDRDDPRYVVTMYEGVHNHVSPGTVYYATHDAASGRFFVAGMHQPGH

SEQ ID NO: 53 - protein, 255 aa, Zea mays
  SITE (1)..(255): GRMZM2G160565_P01 ZmAGL15  corn orthologues preferred
MGRGRVELKRIENKINRQVTFSKRRNGLLKKAYELSVLCDAEVALIIFSSRGKLYEFGSAGITKTLERYQHCCYNAQDSNGALSETQSWYQEMSKLRAKFEALQRTQRHLLGEELGPLSVKELQQLEKQLECALSQARQRKTQLMMEQVEELRRKERHLGEMNRQLKHKLEAEGCSNYRTLQHAAWPAPGSTMVEHDGATYHVHPTTAQSVAMDCEPTLQIGYPPHHQFLPSEAANNIPRSPPGGENNFMLGWVL

SEQ ID NO: 54 - protein, 288 aa, Oryza sativa
  SITE (1)..(288): Os02g47190.1
MFEGMERAGYGVGVGGAGAVGAGVVLSRDPKPRLRWTPDLHERFVEAVTKLGGPDKATPKSVLRLMGMKGLTLYHLKSHLQKYRLGKQNKKDTGLEASRGAFAAHGISFASAAPPTIPSAENNNAGETPLADALRYQIEVQRKLHEQLEVQKKLQMRIEAQGKYLQTILEKAQNNLSYDATGTANLEATRTQLTDFNLALSGFMNNVSQVCEQNNGELAKAISEDNLRTTNLGFQLYHGIQDSDDVKCSQDEGLLLLDLNIKGGGYDHLSSNAMRGGESGLKISQHRR

SEQ ID NO: 55 - protein, 388 aa, Oryza sativa
  SITE (1)..(388): Os03g49880.1
MLPFFDSPSPMDIPLYQQLQLTPPSPKPDHHHHHHSTFFYYHHHPPPSPSFPSFPSPAAATIASPSPAMHPFMDLELEPHGQQLAAAEEDGAGGQGVDAGVPFGVDGAAAAAAARKDRHSKISTAGGMRDRRMRLSLDVARKFFALQDMLGFDKASKTVQWLLNMSKAAIREIMSDDASSVCEEDGSSSLSVDGKQQQHSNPADRGGGAGDHKGAAHGHSDGKKPAKPRRAAANPKPPRRLANAHPVPDKESRAKARERARERTKEKNRMRWVTLASAISVEAATAAAAAGEDKSPTSPSNNLNHSSSTNLVSTELEDGSSSTRHNGVGVSGGRMQEISAASEASDVIMAFANGGAYGDSGSYYLQQQHQQDQWELGGVVYANSRHYC

SEQ ID NO: 56 - protein, 279 aa, Oryza sativa
  SITE (1)..(279): Os09g23620.1
MGRQPCCDKVGLKKGPWTAEEDQKLVSFLLGNGQCCWRAVPKLAGLLRCGKSCRLRWTNYLRPDLKRGLLSETEEKTVIDLHEQLGNRWSKIASHLPGRTDNEIKNHWNTHIKKKLRKMGIDPVTHKPLYPAPPLADGGSPEQKVPEEEEEVEEKSSAAVESSTSTCAGHDVFCTDEVPMLHLDDIVLPPPCDVVGDTAGSPAESSSTSTSSSGGGGIDEEWLLPIMEWPESMYLMGLDDVDMVTTAAPAMATSWEFEDPFNAYQRIALFDHHHELTWA

SEQ ID NO: 57 - protein, 578 aa, Oryza sativa
  SITE (1)..(578): Os03g51330.1
MAYMCADSGNLMAIAQQVIQQQQQQQQQQQRHHHHHHLPPPPPPQSMAPHHHQQKHHHHHQQMPAMPQAPPSSHGQIPGQLAYGGGAAWPAGEHFFADAFGASAGDAVFSDLAAAADFDSDGWMESLIGDAPFQDSDLERLIFTTPPPPVPSPPPTHAAATATATAATAAPRPEAAPALLPQPAAATPVACSSPSPSSADASCSAPILQSLLSCSRAAATDPGLAAAELASVRAAATDAGDPSERLAFYFADALSRRLACGTGAPPSAEPDARFASDELTLCYKTLNDACPYSKFAHLTANQAILEATGAATKIHIVDFGIVQGIQWAALLQALATRPEGKPTRIRITGVPSPLLGPQPAASLAATNTRLRDFAKLLGVDFEFVPLLRPVHELNKSDFLVEPDEAVAVNFMLQLYHLLGDSDELVRRVLRLAKSLSPAVVTLGEYEVSLNRAGFVDRFANALSYYRSLFESLDVAMTRDSPERVRVERWMFGERIQRAVGPEEGADRTERMAGSSEWQTLMEWCGFEPVPLSNYARSQADLLLWNYDSKYKYSLVELPPAFLSLAWEKRPLLTVSAWR

SEQ ID NO: 58 - protein, 239 aa, Oryza sativa
  SITE (1)..(239): Os07g22770.1
MAEHFSHAGMYIGYTADAAASSSSSSSSSSSSSSEMLRFDTGWPDETPAPSSVAGRRRSAGGDHRQGRGQTEAAAAFIGVRRRPWGRFAAEIRDSTRNGARVWIGTFDSAEAAAMAYDQAALSARGAAAALNFPVERVRESLHALSLGAAGGSPVLALKRRHSKRKRRKKAELLAAAAATAATANATPQTRRISKSTELTTATTDEQKRFVVELEDLGAEYLEELLWLSEINGGSDPAD

SEQ ID NO: 59 - protein, 515 aa, Oryza sativa
  SITE (1)..(515): Os05g48990.1
MASPNRHWPSMFRSNLACNIQQQQQPDMNGNGSSSSSFLLSPPTAATTGNGKPSLLSSGCEEGTRNPEPKPRWNPRPEQIRILEGIFNSGMVNPPRDEIRRIRLQLQEYGQVGDANVFYWFQNRKSRTKNKLRAAGHHHHHGRAAALPRASAPPSTNIVLPSAAAAAPLTPPRRHLLAATSSSSSSSDRSSGSSKSVKPAAAALLTSAAIDLFSPAPAPTTQLPACQLYYHSHPTPLARDDQLITSPESSSLLLQWPASQYMPATELGGVLGSSSHTQTPAAITTHPSTISPSVLLGLCNEALGQHQQETMDDMMITCSNPSKVFDHHSMDDMSCTDAVSAVNRDDEKARLGLLHYGIGVTAAANPAPHHHHHHHHLASPVHDAVSAADASTAAMILPFTTTAAATPSNVVATSSALADQLQGLLDAGLLQGGAAPPPPSATVVAVSRDDETMCTKTTSYSFPATMHLNVKMFGEAAVLVRYSGEPVLVDDSGVTVEPLQQGATYYVLVSEEAVH

SEQ ID NO: 60 - protein, 318 aa, Oryza sativa
  SITE (1)..(318): Os04g46240.1
MDFPGDAEDFALEFIREHLLGGDAPVLPPAAVPAAAAYLPTSTMFLPQQQRGYAGLTPQEYVVDSAPAADQAAFRDDQPDPAADVMIMFGGERFPAVKPSSSSSPSLTVTVPPSSFGSWAPAAVPAVAATAAAVEDFRKYRGVRQRPWGKFAAEIRDPKRRGSRVWLGTYDTPVEAARAYDRAAFRMRGAKAILNFPNEVGTRGAELWATPPPTNKRKRQPEDDTAAADDVEVIGVANKAVKTEAPTSAYSSSSLSSMSRDTTATTSSAGTSTGSSEPTSFPVVTPSSWSWDQYWDGLPPLSPLSPHPALGFPQLTVS

SEQ ID NO: 61 - protein, 410 aa, Oryza sativa
  SITE (1)..(410): Os01g74140.1
MDGAMQESREYWRDGGDVVGEELLREILDETAAVHSNSNSNSNSNSNSKEAEEEDEREYFAAAAADEQLQVEAPCGRRRRESMVNKLISTVYSGPTISDIESALSFTAAGDHQLLADGHNFAASSCSPVVFSPEKTLSKTMENKYTLKMKSCGNNGGLADDGYKWRKYGQKSIKNSPNPRSYYRCTNPRCNAKKQVERAVDEPDTLIVTYEGLHLHYTYSHFLHSTSSSSSSTTTQQQLQPQPQMMTNCKKKPKLHLHPLLHDDPPPPPPPPEMTTMMIMQSFSIQQQQHDDDQLLQPAADDHLMVQAPPDDCYNINGSSSSGLMMSLDDDEQAAGAGGLLEDVVPLLVRRPPPPICNNNNYYYSPATTCTSDNEYGSSASASPSSSVSVSSWTTPMSPCIDMAILSNIF

SEQ ID NO: 62 - protein, 233 aa, Oryza sativa
  SITE (1)..(233): Os10g39130.1
MVRGRTELKRIENPTSRQVTFSKRRNGLLKKAFELSVLCDAEVALIVFSPRGRLYEFASAPSLQKTIDRYKAYTKDHVNNKTIQQDIQQVKDDTLGLAKKLEALDESRRKILGENLEGFSIEELRGLEMKLEKSLHKIRLKKTELLEQQIAKLKEKERTLLKDNENLRGKHRNLEAAALVANHMTTTTAPAAWPRDVPMTSSTAGAADAMDVETDLYIGLPGTERSSNRSETG

SEQ ID NO: 63 - protein, 251 aa, Oryza sativa
  SITE (1)..(251): Os09g36730.1
MGRSPCCEKAHTNKGAWTKEEDDRLIAYIKAHGEGCWRSLPKAAGLLRCGKSCRLRWINYLRPDLKRGNFTEEEDELIIKLHSLLGNKWSLIAGRLPGRTDNEIKNYWNTHIRRKLLSRGIDPVTHRPINDSASNITISFEAAAAAARDDKAAVFRREDHPHQPKAVTVAQEQQAAADWGHGKPLKCPDLNLDLCISLPSQEEPMMMKPVKRETGVCFSCSLGLPKSTDCKCSSFLGLRTAMLDFRSLEMK

SEQ ID NO: 64 - protein, 221 aa, Oryza sativa
  SITE (1)..(221): Os05g46020.1
MAAVGAHAAVYHHPVSGLSAPAGDAAYSMSSYFSHGGSSTSSSASSFSAALAAATTPPLPDPSGSQFDISEFFFDDAPPAAVFNGAPTAALPDGAAANATRSAAEAVPAPAPAAVERPRTERIAFRTKSEIEILDDGYKWRKYGKKSVKNSPNPRNYYRCSTEGCNVKKRVERDKDDPSYVVTTYEGTHNHVSPSTVYYASQDAASGRFFVAGTQPPGSLN

SEQ ID NO: 65 - protein, 250 aa, Oryza sativa
  SITE (1)..(250): Os02g45770.1
MGRGRVELKRIENKINRQVTFSKRRNGLLKKAYELSVLCDAEVALIIFSSRGKLYEFGSAGITKTLERYQHCCYNAQDSNNALSETQSWYHEMSKLKAKFEALQRTQRHLLGEDLGPLSVKELQQLEKQLECALSQARQRKTQLMMEQVEELRRKERQLGEINRQLKHKLEVEGSTSNYRAMQQASWAQGAVVENGAAYVQPPPHSAAMDSEPTLQIGYPHQFVPAEANTIQRSTAPAGAENNFMLGWVL

SEQ ID NO: 66 - protein, 266 aa, Brachypodium sp.
  SITE (1)..(266): Bradi5g20520.1
MFEGSYGGGCAAGAEAALSRDPKQRLRWTPELHRRFVDAVAKLGGPDKATPKSVLRLMGIKGLTLFHLKSHLQKYRMGRQTKKATDLELASSGGFAAGDISFSIGTPRLVPAGDDNREISPTDTLRYQIQVQRKLHEQLEVQKKLHARIEAQGRYLKAILEKAKKNISVDINGSPNIESTRSQFMDFNLDLLGLMDNGTQMYEENSEQLMKAISDNNLKDNNLDFQLYDVGSQEAKNVRCTPRTEDLLLLDLNIKGGHDLSSTGMQ

SEQ ID NO: 67 - protein, 385 aa, Brachypodium sp.
  SITE (1)..(385): Bradi1g11060.1
MFPLCDSPSPMDLPLYQQLQLSPPSLKPDPEDHHHQSSFFYYHSSPAFAGADAAFHHSCYLDPGAATLPSAEIDCSPPPELSLMDQALPAAGNTAQGTEHHGSGSGSGVGALESRAAAAARKDRHSKICTAGGMRDRRMRLSLDVARKFFALQDMLGFDKASKTVQWLLNTSKSAIREVMATDDMDPAHSSECEDDDGSSISLSNMPAPEKKGDRGEGKKPATARAARRAANLPKPSRKSGGANAHTIPDKESRTKARERARERTKEKNRMRWVTLASTINLESAARDDELIMASPNNNLNRSSSSSMNTAASADKLEERCCTNGGRTVQEASIASHAIMAGAFGNGGTYGSGSSSGSNYYYQHQQLEEQQWELGGVVFANSRLY

SEQ ID NO: 68 - protein, 269 aa, Brachypodium sp.
  SITE (1)..(269): Bradi4g29800.1
MGRQPCCDKVGLKKGPWTAEEDQKLVSFLLSNGQCCWRAVPKLAAGLLRCGKSCRLRWTNYLRPDLKRGLLSESEEKTVIELHAELGNRWSKIASHLPGRTDNEIKNHWNTHIKKKLKKMGIDPATHKPLLHSASAPPPPPQQNSLEEKATAVVAATTGRVQDEFCADDAIMQLLDDIILPCDVVVAPAPLDTTDSSPAESSSSSSSLGGGFEDDCWLPQMMEWPAESMYLMGLDDMVTGPAAASAWEFEDPFNTYQRIALFDHQDTWA

SEQ ID NO: 69 - protein, 541 aa, Brachypodium sp.
  SITE (1)..(541): Bradi1g10330.1
MAYMCADSGNLMAIAQQVIQQQQQQQQHHQQRHHHHLAPPMPSAPAPPHAQIPASLPYGGASAGWPQAEHFFSDVFGASAADAVFSDLATAADFDSDGWMESLIGDAPVFQDSDLDRLIFTTPPPPVPPPAEPAAAQAENAPASLPLAAATTPVACSPASSSDTSCSAPILQSLLACSRAAAANSGLAATELAKVRAVATDSGDPAERVAFYFSDALARRLACGGAASPVTAADARFAADELTLCYKTLNDACPYSKFAHLTANQAILEATGAATKIHIVDFGIVQGIQWAALLQALATRPEGKPSRIRISGVPSPFLGPEPAASLAATSARLRDFAKLLGVDFEFVPLLRPVDELDQSDFLIEPDEVVAVNFMLQLYHLLGDSDEPVRRVLRLAKSLHPAVVTLGEYEVSLNRAGFVDRFANALSYYRLVFESLDVAMARDSQERVMMERCMFGERIRRAVGPGEGADRTDRMAGSSEWQTLMEWCGFEPVRLSNYAMSQADLLLWNYDSKYKYSLVELQPAFLSLAWEKRPLLTVSAWR

SEQ ID NO: 70 - protein, 191 aa, Brachypodium sp.
  SITE (1)..(191): Bradi1g00670.1
MLLLDMLSSQAAAPMAESPAPTTPAAPAVKQEEEAAAGNGSGRAFRGVRKRPWGKFAAEIRDSTRNGVRVWLGTFDSPEAAAMAYDQAAFAMRGGAAVLNFPAEQVRRSLEGVAMDDGHGHGPVLALKRRHSMRRRPAAAASGRKAAMSKGPGRSQSQPEGVMELEDLGAEYLEELLGASDSQSLWSHHSV

SEQ ID NO: 71 - protein, 271 aa, Brachypodium sp.
  SITE (1)..(271): Bradi1g63680.1
MVNPPKDETVRIRKLLERFGAVGDANVFYWFQNRRSRSRRRQRQMQAAAAAAAAANNNNTSSAAAASATIGGQLPSAMAIVGGSACQYEQQASSSSSSGSTGGSSSLGLFAHGAAGVSSSGPGAGVGYQQLLQQQQAASCGASLSALANSGLMVGDVGDSGGGDDLFAISRQMGFVDHSPVGSSNSSAAPSTAVQQQQQYFSCQLPTATITVFINGVPMEVPRGPIDLRAMFGQDVVLVHSTGALLPVNDYGILIQSLQMGESYFLVARQT

SEQ ID NO: 72 - protein, 364 aa, Brachypodium sp.
  SITE (1)..(364): Bradi5g17490.1
MDFAGEMDDFALEIIREHLLGGDGSTTTAVVPATAADNNHEFPDGGVTFPVPQPPSAAPEPAYLQPAMSFFPQEEQLQGRPYAYTDLTQEYVNSSPAGADMGEVVTFRAPEPVMIQFGGEPSPVATTARAPPSSLTISLPRSSGGSFGWPPRAQQAAAAAAGAAAAPDCQDFRKYRGVRQRPWGKFAAEIRDPKRRGSRVWLGTYDTSVEAARAYDRAAFRMRGAKAILNFPNEVGTRGAELWAPPPPPAPTSQAATGTTNKRKRSQDQEEYCYPEVEAANKAVKTEAAPSPSADTPSSVSREMSTGTACSTVTSAATSEGGFPPLTPSSSGWEQYWEALLGGMPLLSPLSPHPALGFPQLTVS

SEQ ID NO: 73 - protein, 274 aa, Brachypodium sp.
  SITE (1)..(274): Bradi1g59180.1
MAGAGGGEWPFSGDEDSSALLAELGWAASFVVDDCTLQLPPLELHPPPPDEGGGAAASSSSIDDGAATPEPTAGADGKPATGATEAASKPAPAPGRKGQNNGNKRARQPRFAFMTKTEIDHLEDGYRWRKYGQKAVKNSPFPRSYYRCTNSKCTVKKRVERSSNDPSIVITTYEGQHCHHTVTFPRHHFSPHHGHHLLYNDEHPLPPMHGCSSSSSSLFCRPSPSSSSSSLLQQLHCNRQELQAAASYTTASSTVPAVDKGLLDDMVPPGMRHG

SEQ ID NO: 74 - protein, 227 aa, Brachypodium sp.
  SITE (1)..(227): Bradi3g32090.1
MVRGKTELKRIENTTSRQVTFSKRRNGLLKKAFELSVLCDAEVALVVFSPRGRLYEFASSASLQKTIDRYKAYTKDNVNKKTAQQDIQQIRADTVGLAKKLEALEDSKRKILGENLGECTTQELHILEAKIEKSLHIIRAKKSQLLERQIAKLKEKETMLLKDNEELREKQQHLAALMVVPSLNHVALSPLQPEPEPEPSSDAIDTVETELYIGLPGRERSSNRQSG

SEQ ID NO: 75 - protein, 317 aa, Brachypodium sp.
  SITE (1)..(317): Bradi3g46910.1
MGRSPCCEKEAGLKKGPWTPEEDQKLLSHIEQHGHGCWRSLPAKAGLRRCGKSCRLRWTNYLRPDIKRGKFTLQEEQSIIQLHALLGNRWSAIATHLPKRTDNEIKNYWNTHLKKRLAKMGIDPVTHKPRADADAGSGTGARSRVAAHLSHTAQWESARLEAEARLAREAKLRALASPQPAPASAASVLDSPTSTLSFSESALFGAGAARTPVQPVQSYGDACEEQQRFGGGGGETGFAGSGITFASVLLDCSVAAGTEQRLLKMAEDEVGELEDKGYWSSILNMVNSSMSSSLTSEVAADDPEMYLPAAAAAAGEF

SEQ ID NO: 76 - protein, 243 aa, Brachypodium sp.
  SITE (1)..(243): Bradi2g18530.1
MAAVGAAPVLFYQQPAPAAAPALAAGDAIGCFFSPSSPMSSFFSSHGHGGSSSTAGSSPASGFSPALPTQPPPVTDPAAQFDISEYLFDDGIFAAATDAAAPPSGAAVAAAMDGVGASAVAALGRSPADQQQQQAAVERPRTERIAFRTRSEIEILDDGYKWRKYGKKSVKNSPNPRNYYRCSTEGCSVKKRVERDRDDPSYVVTTYEGTHSHVSPSTVYYASQDAASGRFFVAGTQPPGSLH

SEQ ID NO: 77 - protein, 261 aa, Brachypodium sp.
  SITE (1)..(261): Bradi3g51800.2
MGRGRVELKRIENKINRQVTFSKRRNGLLKKAYELSVLCDAEVALIIFSSRGKLYEFGSAGTTKTLERYQHCCYNAQDSNSALSETQSWYQEMSKLKAKLEALQRTQRHLLGEDLGPLSVKELQQLEKQLECSLSQARQRKTQLMMEQVEELRRKERHLGEINRQLKHKLDSEGSSSNNNYRAMQQVSWAAGAVVDEAGAAAYHVQQQQPPHHSAAMDCEPTLQIGYPHQFVTAPEAAANNIPRSSAPAGGENNFMLGWVL

SEQ ID NO: 78 - DNA, 2720 bp, artificial; identical to SEQ ID NO: 30
  expression cassette

SEQ ID NO: 79 - DNA, 3005 bp, artificial; identical to SEQ ID NO: 31
  expression cassette

SEQ ID NO: 80 - DNA, 2774 bp, artificial; identical to SEQ ID NO: 32
  expression cassette

SEQ ID NO: 81 - DNA, 3554 bp, artificial; identical to SEQ ID NO: 33
  expression cassette

SEQ ID NO: 82 - DNA, 2582 bp, artificial; identical to SEQ ID NO: 34
  expression cassette

SEQ ID NO: 83 - DNA, 3062 bp, artificial; identical to SEQ ID NO: 35
  expression cassette

SEQ ID NO: 84 - DNA, 2774 bp, artificial; identical to SEQ ID NO: 36
  expression cassette

SEQ ID NO: 85 - DNA, 2750 bp, artificial; identical to SEQ ID NO: 37
  expression cassette

SEQ ID NO: 86 - DNA, 2570 bp, artificial; identical to SEQ ID NO: 38
  expression cassette

SEQ ID NO: 87 - DNA, 2900 bp, artificial; identical to SEQ ID NO: 39
  expression cassette

SEQ ID NO: 88 - DNA, 2510 bp, artificial; identical to SEQ ID NO: 40
  expression cassette

SEQ ID NO: 89 - DNA, 2732 bp, artificial
  expression cassette
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcatcccccctccccctccgccgccgccgcgccggtaaccaccccgcccctatcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatgggcaggggcaagattgagattaagcggattgagaacgcgaactccaggcaggttacattcagcaagcggcggtcggggctcctcaagaaggcccgggagctgtccgtcctctgcgacgctgaggtggcggtcatcgttttcagcaagtcgggcaagctgttcgagtactccagcacagggatgaagcagactctcagcaggtacggcaaccaccagtcgtcttcagcttcgaaggccgaggaggattgcgcggaggtcgacatcctgaaggatcagctgtctaagctccaggagaagcacctccagctgcagggcaaggggctgaatccgctcaccttcaaggagctgcagtccctggagcagcagctgtaccatgctctcattaccgttagggagcggaaggagcgcctcctgacgaaccagctggaggagagccggctcaaggagcagagggctgagctggagaatgagacactccgcaggcaggtgcaggagctgaggtccttcctcccaagcttcactcactacgtccctagctacatcaagtgcttcgcgattgacccgaagaacgctctgattaatcatgattctaagtgctcactccagaacaccgactcggataccacgctccagctgggcctcccaggggaggctcatgacaggaggacgaacgagggggagagggagtcgccctccagcgattctgttacaactaatacctcgtctgagacggctgagaggggcgaccagtcatccctcgccaactcaccaccagaggctaagaggcagaggttctccgtgtgaaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg